SEQUENCE LISTING (167 sequences)

SEQ ID NO: 1 - protein, 164 aa, Mus sp.
KEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVA

SEQ ID NO: 2 - protein, 50 aa, synthetic construct
KEICGNPVTDNVKDITKLVANLPNDYMITLNYVAGMDVLPSHCWLRDMVI

SEQ ID NO: 3 - protein, 50 aa, synthetic construct
FFSIFNRSIDAFKDFMVASDTSDCVLSSTLGPEKDSRVSVTKPFMLPPVA

SEQ ID NO: 4 - protein, 30 aa, synthetic construct
IIHKLGIIVDDLFFCMEENAPKNIKEFPKR

SEQ ID NO: 5 - protein, 29 aa, synthetic construct
  VARIANT 4: note = E, D or K
  VARIANT 9: note = V or E
  VARIANT 11: note = D, Y, E or F
  VARIANT 13: note = F or W
  VARIANT 15: note = C or D
  VARIANT 18: note = E or D
  VARIANT 19: note = N, E, Q or D
  VARIANT 20: note = A or P
  VARIANT 21: note = P, D, K or V
  VARIANT 23: note = N, I or K
  VARIANT 24: note = I, K, D, N or E
  VARIANT 25: note = K, D, E, I, W, T or Y
  VARIANT 26: note = E, F or V
  VARIANT 27: note = F, Y, D, or E
  VARIANT 28: note = P, K, F or Y
  VARIANT 29: note = K, R or P
IIHXLGIIXDXLXFXMEXXXXKXXXXXXX

SEQ ID NO: 6 - protein, 7 aa, synthetic construct
DRTTLTN

SEQ ID NO: 7 - protein, 7 aa, synthetic construct
RRRRRRR

SEQ ID NO: 8 - protein, 5 aa, synthetic construct
GGGGS

SEQ ID NO: 9 - protein, 30 aa, synthetic construct
MKEICGNPVTDNVKDITKLVANLPNDYMIT

SEQ ID NO: 10 - protein, 30 aa, synthetic construct
ITKLVANLPNDYMITLNYVAGMDVLPSHCW

SEQ ID NO: 11 - protein, 30 aa, synthetic construct
LNYVAGMDVLPSHCWLRDMVIQLSLSLTTL

SEQ ID NO: 12 - protein, 30 aa, synthetic construct
LRDMVIQLSLSLTTLLDKFSNISEGLSNYS

SEQ ID NO: 13 - protein, 30 aa, synthetic construct
LDKFSNISEGLSNYSIIDKLGKIVDDLVLC

SEQ ID NO: 14 - protein, 30 aa, synthetic construct
IIDKLGKIVDDLVLCMEENAPKNIKESPKR

SEQ ID NO: 15 - protein, 30 aa, synthetic construct
MEENAPKNIKESPKRPETRSFTPEEFFSIF

SEQ ID NO: 16 - protein, 30 aa, synthetic construct
PETRSFTPEEFFSIFNRSIDAFKDFMVASD

SEQ ID NO: 17 - protein, 30 aa, synthetic construct
NRSIDAFKDFMVASDTSDCVLSSTLGPEKD

SEQ ID NO: 18 - protein, 29 aa, synthetic construct
TSDCVLSSTLGPEKDSRVSVTKPFMLPPV

SEQ ID NO: 19 - protein, 30 aa, synthetic construct
LDKASNISEGLSNYSIIHKLGIIVDDLFFC

SEQ ID NO: 20 - protein, 30 aa, synthetic construct; identical to SEQ ID NO: 4

SEQ ID NO: 21 - protein, 30 aa, synthetic construct
IIHELGIIVDDLFFCMEENADKNIDEFPKR

SEQ ID NO: 22 - protein, 29 aa, synthetic construct
IIHKLGIIVDYLFFCMEENAPKNIKEYPK

SEQ ID NO: 23 - protein, 29 aa, synthetic construct
IIHKLGIIVDELFFCMEEEPKNIKEFDKR

SEQ ID NO: 24 - protein, 30 aa, synthetic construct
IIHKLGIIVDDLFFCMEENAPKNDKEDPKR

SEQ ID NO: 25 - protein, 31 aa, synthetic construct
IIHKLGIIVDDLFFCMEENAVPKNIVEFPKR

SEQ ID NO: 26 - protein, 30 aa, synthetic construct
IIHDLGIIVDDLFFCMEENAPKNEKEFYKR

SEQ ID NO: 27 - protein, 30 aa, synthetic construct
IIHKLGIIVDFLFFCMEENAPKNIWEFPKY

SEQ ID NO: 28 - protein, 30 aa, synthetic construct
IIHKLGIIVDELFFCMEENAPKNIEEFPKR

SEQ ID NO: 29 - protein, 30 aa, synthetic construct
IIHKLGIIVDDLFFCMEENAPKNITEFPKR

SEQ ID NO: 30 - protein, 30 aa, synthetic construct
IIHKLGIIVDDLFFCMEEQAPKNIDEFPKR

SEQ ID NO: 31 - protein, 30 aa, synthetic construct
IIHELGIIVDELFFCMEEDAPKNIEEFPKR

SEQ ID NO: 32 - protein, 30 aa, synthetic construct
IIHDLGIIVDYLFFCMEEDAPKNIWEFPKR

SEQ ID NO: 33 - protein, 30 aa, synthetic construct
IIHELGIIVDFLFFCMEEDAPKNIYEFPKR

SEQ ID NO: 34 - protein, 30 aa, synthetic construct
IIHELGIIVDELFFCMEEQAPKNIEEFPKR

SEQ ID NO: 35 - protein, 30 aa, synthetic construct
IIHELGIIVDELFFCMEENAPKNIDEFPKR

SEQ ID NO: 36 - protein, 30 aa, synthetic construct
IIHELGIIEDDLWFDMEDNAPKNIEEFPKR

SEQ ID NO: 37 - protein, 51 aa, synthetic construct
WISEMVVQLSDSLTDLLDKFSNISEGLSNYSIIDKLVNIVDDLVECVKENS

SEQ ID NO: 38 - protein, 51 aa, synthetic construct
WLRDMVIQLSLSLTTLLDKFSNISEGLSNYSIIDKLGKIVDDLVLCMEENA

SEQ ID NO: 39 - protein, 51 aa, synthetic construct
WISEMVVQLSDSLTDLLDKASNISEGLSNYSIIHKLVIIVDDLFFCVKENS

SEQ ID NO: 40 - protein, 51 aa, synthetic construct
WLRDMVIQLSLSLTTLLDKASNISEGLSNYSIIHKLGIIVDDLFFCMEENA

SEQ ID NO: 41 - protein, 51 aa, synthetic construct
WISEMVVQLSDSLTDLLDKASNISEGLSNYSIIHKLVNIVDDLFECVKENS

SEQ ID NO: 42 - protein, 51 aa, synthetic construct
WISEMVVQLSDSLTDLLDKFSNISEGLSNYSIIDKLVIIVDDLVFCVKENS

SEQ ID NO: 43 - protein, 51 aa, synthetic construct
WISEMVVQISLSLTTLLDKFSNISEGLSNYSIIDKLVKIVDDLVLCVKENS

SEQ ID NO: 44 - protein, 164 aa, synthetic construct; identical to SEQ ID NO: 1

SEQ ID NO: 45 - protein, 164 aa, synthetic construct
KEICGAPVTDNVKDITKLVANLPNDYMITLNYVAGMDVLPSHCWLRDMVIQLSLSLTTLLDKASNISEGLSNYSIIHKLGIIVDDLFFCMEENAPKNIKEFPKRPETRSFTPEEFFSIFNRSIDAFKDFMVASDTSDCVLSSTLGPEKDSRVSVTKPFMLPPVA

SEQ ID NO: 46 - protein, 165 aa, synthetic construct
MKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVA

SEQ ID NO: 47 - protein, 165 aa, synthetic construct
MKEICGAPVTDNVKDITKLVANLPNDYMITLNYVAGMDVLPSHCWLRDMVIQLSLSLTTLLDKASNISEGLSNYSIIHKLGIIVDDLFFCMEENAPKNIKEFPKRPETRSFTPEEFFSIFNRSIDAFKDFMVASDTSDCVLSSTLGPEKDSRVSVTKPFMLPPVA

SEQ ID NO: 48 - protein, 177 aa, synthetic construct
MDRTTLTNGGGGSKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVA

SEQ ID NO: 49 - protein, 177 aa, synthetic construct
MRRRRRRRGGGGSKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVA

SEQ ID NO: 50 - protein, 177 aa, synthetic construct
MDRTTLTNGGGGSKEICGAPVTDNVKDITKLVANLPNDYMITLNYVAGMDVLPSHCWLRDMVIQLSLSLTTLLDKASNISEGLSNYSIIHKLGIIVDDLFFCMEENAPKNIKEFPKRPETRSFTPEEFFSIFNRSIDAFKDFMVASDTSDCVLSSTLGPEKDSRVSVTKPFMLPPVA

SEQ ID NO: 51 - protein, 13 aa, synthetic construct
ENAPKNITEFPKR

SEQ ID NO: 52 - protein, 10 aa, synthetic construct
PKNITEFPKR

SEQ ID NO: 53 - protein, 5 aa, synthetic construct
IHKLG

SEQ ID NO: 54 - protein, 5 aa, synthetic construct
IIVDD

SEQ ID NO: 55 - protein, 5 aa, synthetic construct
DDLFF

SEQ ID NO: 56 - protein, 5 aa, synthetic construct
KNITE

SEQ ID NO: 57 - protein, 5 aa, synthetic construct
NITEF

SEQ ID NO: 58 - protein, 30 aa, synthetic construct
  SITE 16: note = Norleucine
IIHKLGIIVDDLFFCXEENAPKNIKEFPKR

SEQ ID NO: 59 - protein, 30 aa, synthetic construct
  SITE 16: note = Norleucine
IIHKLGIIVDDLFFCXEENAPKNITEFPKR

SEQ ID NO: 60 - protein, 5 aa, synthetic construct
IHKLG

SEQ ID NO: 61 - protein, 5 aa, synthetic construct
IIVDD

SEQ ID NO: 62 - protein, 5 aa, synthetic construct
DDLFF

SEQ ID NO: 63 - protein, 5 aa, synthetic construct
  SITE 4: note = Aspartic acid beta-methyl ester
IIVDD

SEQ ID NO: 64 - protein, 5 aa, synthetic construct
  SITE 5: note = Aspartic acid beta-methyl ester
IIVDD

SEQ ID NO: 65 - protein, 5 aa, synthetic construct
  SITE 4: note = Aspartic acid beta-methyl ester
  SITE 5: note = Aspartic acid beta-methyl ester
IIVDD

SEQ ID NO: 66 - protein, 30 aa, synthetic construct; identical to SEQ ID NO: 58
  SITE 16: note = Norleucine

SEQ ID NO: 67 - protein, 30 aa, synthetic construct; identical to SEQ ID NO: 59
  SITE 16: note = Norleucine

SEQ ID NO: 68 - protein, 5 aa, synthetic construct
IHKLG

SEQ ID NO: 69 - protein, 5 aa, synthetic construct
IIVDD

SEQ ID NO: 70 - protein, 5 aa, synthetic construct
DDLFF

SEQ ID NO: 71 - protein, 5 aa, synthetic construct
IHKLG

SEQ ID NO: 72 - protein, 5 aa, synthetic construct
IIVDD

SEQ ID NO: 73 - protein, 5 aa, synthetic construct
DDLFF

SEQ ID NO: 74 - protein, 164 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 75 - protein, 164 aa, synthetic construct
KEICGAPVTDNVKDITKLVANLPNDYMITLNYVAGMDVLPSHCWLRDMVIQLSLSLTTLLDKASNISEGLSNYSIIHKLGKIVDDLFFCMEENAPKNIKEFPKRPETRSFTPEEFFSIFNRSIDAFKDFMVASDTSDCVLSSTLGPEKDSRVSVTKPFMLPPVA

SEQ ID NO: 76 - protein, 146 aa, synthetic construct
ICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEK

SEQ ID NO: 77 - protein, 146 aa, synthetic construct
ICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEK

SEQ ID NO: 78 - protein, 30 aa, synthetic construct
IIDKLYYIVDHLVLCMEENAPKNIKERPKR

SEQ ID NO: 79 - protein, 30 aa, synthetic construct
IITKLGKIVDDLVLCMEENWDKNDKESFKR

SEQ ID NO: 80 - protein, 30 aa, synthetic construct
IIDKLGKIVQDLVSCMEEWAMINIKESPKR

SEQ ID NO: 81 - protein, 30 aa, synthetic construct
IIDKLGFIVDGLVLCMEENAPKNIKESPGR

SEQ ID NO: 82 - protein, 30 aa, synthetic construct
IIDKLGKIHDDLVLCMEENAPTTIKESPKR

SEQ ID NO: 83 - protein, 30 aa, synthetic construct; identical to SEQ ID NO: 4

SEQ ID NO: 84 - protein, 30 aa, synthetic construct
IIDKLGKKVDDLYLYMEELAPKNITEGPKR

SEQ ID NO: 85 - protein, 30 aa, synthetic construct
IIYSLGKIVDDLVLCMEEPAPDNIKESWKR

SEQ ID NO: 86 - protein, 30 aa, synthetic construct
IIEKLGSGVDDLVLCMEENAVKNMKSSPKR

SEQ ID NO: 87 - protein, 30 aa, synthetic construct
EIDKKGKIDDDLVLFSEENAPKNIKESPKR

SEQ ID NO: 88 - protein, 30 aa, synthetic construct
IIWTLGKIVDLLHLCMEENAPKNISESPKR

SEQ ID NO: 89 - protein, 30 aa, synthetic construct
IIGKCGKISRDLVLCMEENAPKNEKESKKR

SEQ ID NO: 90 - protein, 30 aa, synthetic construct
IIDKLGKICDDLVLWMEENAPANIKESPKR

SEQ ID NO: 91 - protein, 30 aa, synthetic construct
IIDKLLKIVDDLVDCMELWAPKNIKESPKR

SEQ ID NO: 92 - protein, 30 aa, synthetic construct
IIDKWGKIVDDLPLCMEYNAIKQIKESPKR

SEQ ID NO: 93 - protein, 30 aa, synthetic construct
IIDKLGKIVMDLVNCMEENAMKNIKESPTR

SEQ ID NO: 94 - protein, 30 aa, synthetic construct
IIDKLGSIVDDLVVCMEENAPKNIKESPKL

SEQ ID NO: 95 - protein, 30 aa, synthetic construct
IIDKLGVIVDDLVLCMEENAPKSIKESPKR

SEQ ID NO: 96 - protein, 30 aa, synthetic construct
IIDKCGKCVDDVVLCMESNAPKNIKRSDKR

SEQ ID NO: 97 - protein, 30 aa, synthetic construct
IIDMLGKIVDDLVCCMEENMPKNIKESPKR

SEQ ID NO: 98 - protein, 30 aa, synthetic construct
IIWKLGKIVDDLVLCMEYNAPKNIKESPKR

SEQ ID NO: 99 - protein, 30 aa, synthetic construct
MIDKLGKIVDDLVLCMEENAPKVIVESPKS

SEQ ID NO: 100 - protein, 30 aa, synthetic construct
ACDKLGKKADDLMLCMEENAPKNIKNSPKR

SEQ ID NO: 101 - protein, 30 aa, synthetic construct
IIDALPKIVDDLVLCMEEYAPKNIKESPKR

SEQ ID NO: 102 - protein, 30 aa, synthetic construct
IIWKLGKIVDDLVLCMEENAPKKTKRSPKR

SEQ ID NO: 103 - protein, 30 aa, synthetic construct
IIDKLGKIWDDLVLCMEMYAPKNIKESPKR

SEQ ID NO: 104 - protein, 30 aa, synthetic construct
FSDKLMKIVHDLVLCMEEPAPKNIKESPKR

SEQ ID NO: 105 - protein, 30 aa, synthetic construct
IVVKLGKIVHDLPQCMEENAPKNIKSSPKR

SEQ ID NO: 106 - protein, 30 aa, synthetic construct
KEDKLGKIVDDLMLCMEENAPKNIFESEPR

SEQ ID NO: 107 - protein, 30 aa, synthetic construct; identical to SEQ ID NO: 27

SEQ ID NO: 108 - protein, 30 aa, synthetic construct
ICDYYHKIVDDLVLCTEENAPKNIKELPKR

SEQ ID NO: 109 - protein, 30 aa, synthetic construct
IKDALGKEVDDLGLYMEENAPKNIKESPMR

SEQ ID NO: 110 - protein, 30 aa, synthetic construct
IIDKLGQIVDDLVCNMEENAPKDIKESPKM

SEQ ID NO: 111 - protein, 30 aa, synthetic construct
IIDELGKIVDDHVLCMEENAPQNIKESPKL

SEQ ID NO: 112 - protein, 30 aa, synthetic construct
IGDKLGDIVDDLVLCMEENAPENIKESPKR

SEQ ID NO: 113 - protein, 30 aa, synthetic construct
IIDKLGKITDDLVLCMEEAAPKNIKEQPKR

SEQ ID NO: 114 - protein, 30 aa, synthetic construct
IIDKLNKIVDLLVLCMEINAPKNIKWSPKR

SEQ ID NO: 115 - protein, 30 aa, synthetic construct
IQDKLVKIVDDLVLCMESNAPKNIKESPKR

SEQ ID NO: 116 - protein, 30 aa, synthetic construct
IIDKMGKIREDLVLCMEENMPKNIKESPKR

SEQ ID NO: 117 - protein, 30 aa, synthetic construct
IIDNLGKIVDLLVLCMEENAPKNIKESPKR

SEQ ID NO: 118 - protein, 30 aa, synthetic construct
IIDKVGKIKDDLVLCMEENAPKNFKESPKR

SEQ ID NO: 119 - protein, 30 aa, synthetic construct
IIDSLGNGVDDLGLCMEENAPKNIKESPKR

SEQ ID NO: 120 - protein, 30 aa, synthetic construct
IIDKLLKIVDDLVLCMEEIAPKNIKEFPKR

SEQ ID NO: 121 - protein, 30 aa, synthetic construct
IIDKLQKIVDDLVLCMELNWPKNIKEKPKR

SEQ ID NO: 122 - protein, 30 aa, synthetic construct
IIDYLKKAVDDLVLCMEENAPKNIKESPFR

SEQ ID NO: 123 - protein, 30 aa, synthetic construct
IINKLGKIMDDQVLCMEERAPKLIKESPCR

SEQ ID NO: 124 - protein, 30 aa, synthetic construct
IIDCLGKIVDDLVLEMEENAPKNSKESPLI

SEQ ID NO: 125 - protein, 30 aa, synthetic construct
ICDKLGKIVDDLVLCMEENAPKNIKESPKR

SEQ ID NO: 126 - protein, 30 aa, synthetic construct
IIDFLCKIWDDMVLCMEENAPKNIKESYKR

SEQ ID NO: 127 - protein, 30 aa, synthetic construct
IIDKLGKIVKDLVLCMEEDAPKNIKESPKR

SEQ ID NO: 128 - protein, 30 aa, synthetic construct
KIDKDGKIVDDLVLCMEENAPKFIWESPKR

SEQ ID NO: 129 - protein, 30 aa, synthetic construct
PIDKLGKINDDLVLCMEENAPKNTKESPKR

SEQ ID NO: 130 - protein, 30 aa, synthetic construct
IKLKLGKITDDLVCCMEEMAPKNIKESPKR

SEQ ID NO: 131 - protein, 30 aa, synthetic construct
FIDKLVKIEDDLVLCMEENAPKNIKESPKA

SEQ ID NO: 132 - protein, 30 aa, synthetic construct
IIDGLGKIKDDLALCMEYNAPKNIIESPKR

SEQ ID NO: 133 - protein, 30 aa, synthetic construct
IIDKEGKKVDDLVLCMEENAPKNIKESPNK

SEQ ID NO: 134 - protein, 30 aa, synthetic construct
IIDKLGKIVDDLVLCMEERAPKNIKESPKR

SEQ ID NO: 135 - protein, 30 aa, synthetic construct
MIDKLMKTVDDLVLCMEANAPKDIKESPKR

SEQ ID NO: 136 - protein, 30 aa, synthetic construct
IIDKLGKIVDDLVVCMEENAPKSIKESPKR

SEQ ID NO: 137 - protein, 30 aa, synthetic construct
IIDKLGKIVSDLVLCMEENAPKNIGESPKR

SEQ ID NO: 138 - protein, 30 aa, synthetic construct
IIDKLGKIVTDVVLCMEEFAPKNIKHSPHM

SEQ ID NO: 139 - protein, 30 aa, synthetic construct
IIDKPGNIVDDAVVCMEENAPKNIKESVKR

SEQ ID NO: 140 - protein, 30 aa, synthetic construct
IIDKLRKIVIDLVLCYPGNAVKNIKESPKR

SEQ ID NO: 141 - protein, 30 aa, synthetic construct
IIDKQGKIVLCLVLGMEEWAPKNTKESPKR

SEQ ID NO: 142 - protein, 30 aa, synthetic construct
IIDKWGKIVDDAVHCMEENAPWNIIISPKR

SEQ ID NO: 143 - protein, 30 aa, synthetic construct
IIDQLIKIVDDLVLNMEENAPKNTKESPNR

SEQ ID NO: 144 - protein, 30 aa, synthetic construct
IQDSLGKIVPDLVLCWEENAPKNIKESPKR

SEQ ID NO: 145 - protein, 30 aa, synthetic construct
IIDKLGKIVFLAVLCMEENHPKNIKESPKR

SEQ ID NO: 146 - protein, 30 aa, synthetic construct
LRYALGKIVDDLVLCMEENAPQNIRESPKR

SEQ ID NO: 147 - protein, 30 aa, synthetic construct
IIDELGKIVDDLVQCMEENAPHVIKESPKR

SEQ ID NO: 148 - protein, 30 aa, synthetic construct
IIDKLDTIVDNLVLCMEENAPKNITESPKM

SEQ ID NO: 149 - protein, 30 aa, synthetic construct
IIDLLGRIVDDLDSCMEENPPKNIKESPKR

SEQ ID NO: 150 - protein, 30 aa, synthetic construct
SIDKKGFIVDDLVHCMEENPPKNIHESPKR

SEQ ID NO: 151 - protein, 30 aa, synthetic construct
IIHKQGKIKDGLVLCMCESAPKNIKESPKR

SEQ ID NO: 152 - protein, 30 aa, synthetic construct
RIDKLRKIVHDLVLRMFEGAPKNIKESPKR

SEQ ID NO: 153 - protein, 30 aa, synthetic construct; identical to SEQ ID NO: 29

SEQ ID NO: 154 - protein, 30 aa, synthetic construct
IIDKHGFIVDDPVLCCEENAPKNIKESTKR

SEQ ID NO: 155 - protein, 30 aa, synthetic construct
IIDKLAKIVDDLVFCMEEQAPKNSMESPFR

SEQ ID NO: 156 - protein, 30 aa, synthetic construct
IIDKPGKAVDDLVLCMGENAPKNIKESPKR

SEQ ID NO: 157 - protein, 30 aa, synthetic construct
IIDRNGKIVDDLVLCMEENAPRNIKESPKR

SEQ ID NO: 158 - protein, 30 aa, synthetic construct
IIDKIGKIVDDLCLCMEENAPKNIKESPKR

SEQ ID NO: 159 - protein, 30 aa, synthetic construct
IIDKLGCIVDDLGLCMEENAPKAIKESPKD

SEQ ID NO: 160 - protein, 30 aa, synthetic construct
IIKKLGTIVDDLVLCMEENAPVNNKEAPMR

SEQ ID NO: 161 - protein, 30 aa, synthetic construct
PIAKLGTIVDDNVGCMEENAPKFIKESPKR

SEQ ID NO: 162 - protein, 30 aa, synthetic construct
VIDKLGKIVDPLVLCMLENAPKLIKVSPKR

SEQ ID NO: 163 - protein, 30 aa, synthetic construct
IIDKLGKIVADPVLCMEAYAPKNIKVSPKR

SEQ ID NO: 164 - protein, 30 aa, synthetic construct
IHDKLGKILDDLVHCMEENMLKNIKESPKN

SEQ ID NO: 165 - protein, 30 aa, synthetic construct
IIDKPGKIVDDLVLCMEENQPENIKESPMR

SEQ ID NO: 166 - protein, 30 aa, synthetic construct
FIDKLGKIVDDLVLCMEPNWPKEIKESPKR

SEQ ID NO: 167 - protein, 30 aa, synthetic construct
IIDKLGKIVKQLVLCMWENAPKNTKESPKR

(19) United States
(12) Patent Application Publication
(10) Pub. No.: US 2026/0263610 A1
(43) Pub. Date: Sep. 10, 2026

(54) MURINE STEM CELL FACTOR POLYPEPTIDES, COMPOSITIONS, AND METHODS OF USE

(71) Applicant: ELIXR LTD, Rehovot (IL)

(72) Inventors: Yaron FUCHS, Haifa (IL); Matan YAMPOLSKY, Rehovot (IL); Roi ANKAWA, Hadera (IL); Noa JOSEPH, Ramat Gan (IL); Guy MANN, Haifa (IL); Ronen WEISS, Rehovot (IL); Nimrod KRUPNIK, Alonim (IL); Roy HESSEL, Herzeliya (IL)

(21) Appl. No.: 19/189,124

(22) Filed: Apr. 24, 2025

Related U.S. Application Data
(60) Provisional application No. 63/768,034, filed on Mar. 6, 2025.

(51) Int. Cl. A61K 47/64; A61K 38/22; A61K 38/34; A61P 17/00

(52) U.S. Cl. CPC A61K 47/645; A61K 38/2285; A61K 38/34; A61P 17/00

(57) ABSTRACT

Provided herein are murine stem cell factor (mSCF) polypeptides, as well as compositions comprising the same, and methods of using the same. In some embodiments, the mSCF polypeptides are for use in treating one or more conditions in a human subject. Such conditions to be treated may include, but are not limited to, melanocyte dysfunction or deficiency, and processes associated with the same. The present disclosure also provides the use of such mSCF polypeptides for the treatment of hair loss and/or hair whitening.

### CROSS-REFERENCE TO RELATED APPLICATION

This application claims priority to U.S. Provisional Application Ser. No. 63/768,034, filed on Mar. 6, 2025, the disclosure of which is hereby incorporated by reference in its entirety.

### INCORPORATION OF SEQUENCE LISTING

The instant application contains a Sequence Listing which has been submitted electronically in XML format and is hereby incorporated by reference in its entirety. Said XML Sequence Listing was created on Apr. 16, 2025, is named 229732-601201_SL.xml, and is 159,059 bytes in size.

### BACKGROUND

Hair whitening, hair loss, and skin replenishment are areas of concern in the field of cosmetic dermatology. Melanocytes, responsible for, e.g., hair pigmentation, experience functional decline due to aging, environmental stressors, and genetic factors, leading to hair whitening and hair loss. Current treatments address these symptoms superficially but fail to target the root cause.

### SUMMARY

In view of the foregoing, there is a need for improved treatments (e.g., cosmetic treatments) for conditions relating to deficiency and/or dysfunction in melanocytes and/or melanocyte stem cells. Provided herein, according to various aspects and embodiments, are compositions and methods for addressing this need, and providing further advantages as well. In some embodiments, the present disclosure provides a cosmetic approach to address such concerns by incorporating peptides and compounds that activate melanocyte stem cells and their progeny. This activation is believed to restore melanocyte function, improve hair pigmentation, and stimulate hair and skin regeneration, providing a significant advancement over existing treatments.

In some aspects, the present disclosure relates to compositions for the prevention of hair whitening and hair loss, skin replenishment, as well as skin and hair regeneration. In some embodiments, the present disclosure provides compositions containing compounds and peptides that specifically target and activate key components within the melanocyte lineage, thereby protecting stem cells for apoptosis, increasing stem cell survival and vitality, and/or enhancing melanocyte functionality, thus promoting hair pigmentation, as well as stimulating hair and skin replenishment.

According to various aspects and embodiments, the present disclosure provides murine stem cell factor (mSCF) polypeptides, including mSCF polypeptides for use in treating conditions of a human subject. Melanocyte-stimulating hormone (MSH) and Endothelin polypeptides for treating the same are also provided herein.

In one aspect, provided herein is a method of treating a condition a human subject. In some embodiments, the method comprises administering to the human subject a composition comprising one or more of a mSCF polypeptide, an MSH polypeptide, or an Endothelin polypeptide. In some embodiments, the composition comprises the mSCF polypeptide. In some embodiments, the condition comprises melanocyte deficiency or dysfunction.

In some embodiments, the treating comprises one or more of: (i) inhibiting apoptosis of melanocytes or melanocyte stem cells (McSCs), (ii) increasing number or survival of melanocytes or McSCs, (iii) increasing a level of melanin synthesis, (iv) inhibiting hair whitening, (v) promoting increased hair pigmentation, (vi) inhibiting hair loss, or (vii) promoting increased hair growth; as compared to the absence of said treating. In some embodiments, the administering comprises topical administration of the composition to the skin of the human subject. In some embodiments, the skin comprises the scalp or face of the human subject. In some embodiments, the treating is effective to reduce a rate of hair whitening. In some embodiments, the treating is effective to promote an increase in hair pigmentation in the subject.

In some embodiments, the mSCF polypeptide comprises a polypeptide of SEQ ID NO: 1, or a fragment or variant thereof. In some embodiments, the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2. In some embodiments, the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3. In some embodiments, the mSCF polypeptide comprises a sequence with at least 80% identity to 20 contiguous amino acids of SEQ ID NO: 1. In some embodiments, the mSCF polypeptide comprises (i) a sequence with between 80% to 99% identity to 20 contiguous amino acids of SEQ ID NO: 1, and (ii) one or more amino acid substitutions as compared to said 20 contiguous amino acids of SEQ ID NO: 1. In some embodiments, the one or more amino acid substitutions include one or more of D77H, K78E, K78D, K81I, V83E, D85Y, D85E, D85F, V87F, V87W, L88F, C89D, E92D, N93E, N93Q, N93D, A94P, P95D, P95K, P95V, N97I, N97K, 198K, 198D, 198N, 198E, K99D, K99E, K99I, K99W, K99T, K99Y, E100F, E100V, S101F, S101Y, S101D, S101E, P102K, P102F, P102Y, K103R, K103P, or R104Y, relative to SEQ ID NO: 1.

In some embodiments, the mSCF polypeptide comprises a sequence with at least 80% identity to 20 contiguous amino acids of SEQ ID NO: 4. In some embodiments, the mSCF polypeptide comprises a sequence with at least 80% identity to SEQ ID NO: 4. In some embodiments, the mSCF polypeptide comprises the amino acid sequence of SEQ ID NO: 5. In some embodiments, the mSCF polypeptide comprises a sequence with at least 80% sequence identity to any one of SEQ ID NOs: 15-36, 44, 58, or 59. In some embodiments, the mSCF polypeptide comprises the sequence of any one of SEQ ID NOs: 15-36, 44, 58, or 59. In some embodiments, the mSCF polypeptide comprises a sequence with at least 80% sequence identity to any one of SEQ ID NOs: 78-167. In some embodiments, the mSCF polypeptide comprises the sequence of any one of SEQ ID NOs: 78-167.

In some embodiments, the mSCF polypeptide is at least 20 amino acids in length, such as at least 30 amino acids in length. In some embodiments, the mSCF polypeptide is 20 amino acids to 75 amino acids in length, such as about 30 amino acids in length.

In some embodiments, the mSCF polypeptide comprises at least 4 contiguous amino acids of SEQ ID NO: 29, such as 5 to 13 contiguous amino acids of SEQ ID NO: 29. In some embodiments, the mSCF polypeptide is selected from any one of SEQ ID NOs: 51-57. In some embodiments, the mSCF polypeptide consists of the amino acid sequence according to SEQ ID NO: 54, 55, or 57.

In some embodiments, the mSCF polypeptide comprises one or more of a biotin group, a palmitoyl group, a norleucine, or an aspartic acid methyl ester. In some embodiments, the biotin group is joined to the mSCF polypeptide by a C11 linker.

In some embodiments, mSCF polypeptide is joined to a cell-penetrating peptide, such as a cell-penetrating peptide comprising the sequence of SEQ ID NO: 6 or 7. In some embodiments, the cell-penetrating peptide is located at or within 5 amino acids of the N- or C-terminus. In some embodiments, the mSCF polypeptide is joined to the cell-penetrating peptide by a linker sequence, such as a linker sequence comprising the sequence of SEQ ID NO: 8.

In some embodiments, the composition comprises the MSH polypeptide. In some embodiments, the MSH polypeptide is a β-MSH polypeptide or a γ-MSH polypeptide. In some embodiments, the composition comprises the Endothelin polypeptide. In some embodiments, the Endothelin polypeptide is an Endothelin-1 polypeptide or an Endothelin-3 polypeptide. In some embodiments, the composition comprises histamine. In some embodiments, the composition comprises two or more of the mSCF polypeptide, the MSH polypeptide, the Endothelin polypeptide, or histamine.

In some embodiments, the composition further comprises a delivery vehicle, wherein the delivery vehicle is effective to increase delivery of the mSCF polypeptide across the epidermis of the human subject. In some embodiments, the delivery vehicle comprises microneedles, liposomes, or a chemical penetration enhancer.

In one aspect, provided herein is the use of an mSCF polypeptide in the treatment of a human subject according to any of the methods disclosed herein. The mSCF polypeptide may be an mSCF polypeptide according to any of the various aspects and embodiments disclosed herein, including with respect to the various methods and compositions disclosed herein.

In one aspect, provided herein is a composition comprising an mSCF polypeptide. The mSCF polypeptide may be an mSCF polypeptide according to any of the various aspects and embodiments disclosed herein, including with respect to the various methods disclosed herein. In some embodiments, (i) the mSCF polypeptide is joined to a cell-penetrating peptide, or (ii) the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2. In some embodiments, the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3.

In some embodiments, the mSCF polypeptide comprises a sequence with at least 80% identity to 20 contiguous amino acids of SEQ ID NO: 1. In some embodiments, the mSCF polypeptide comprises (i) a sequence with between 80% to 99% identity to 20 contiguous amino acids of SEQ ID NO: 1, and (ii) one or more amino acid substitutions as compared to said 20 contiguous amino acids of SEQ ID NO: 1. In some embodiments, the one or more amino acid substitutions include one or more of D77H, K78E, K78D, K81I, V83E, D85Y, D85E, D85F, V87F, V87W, L88F, C89D, E92D, N93E, N93Q, N93D, A94P, P95D, P95K, P95V, N97I, N97K, 198K, 198D, 198N, 198E, K99D, K99E, K99I, K99W, K99T, K99Y, E100F, E100V, S101F, S101Y, S101D, S101E, P102K, P102F, P102Y, K103R, K103P, or R104Y, relative to SEQ ID NO: 1. In some embodiments, the mSCF polypeptide comprises a sequence with at least 80% identity to 20 contiguous amino acids of SEQ ID NO: 4. In some embodiments, the mSCF polypeptide comprises a sequence with at least 80% identity to SEQ ID NO: 4. In some embodiments, the mSCF polypeptide comprises the amino acid sequence of SEQ ID NO: 5. In some embodiments, the mSCF polypeptide comprises (i) a sequence with at least 80% sequence identity to any one of SEQ ID NOs: 15-36, 44, 58, or 59; or (ii) the sequence of any one of SEQ ID NOs: 15-36, 44, 58, or 59. In some embodiments, the mSCF polypeptide comprises (i) a sequence with at least 80% sequence identity to any one of SEQ ID NOs: 78-167; or (ii) the sequence of any one of SEQ ID NOs: 78-167. In some embodiments, the mSCF polypeptide is at least 20 amino acids in length, such as at least 30 amino acids in length. In some embodiments, the mSCF polypeptide is 20 amino acids to 75 amino acids in length, such as about 30 amino acids in length. In some embodiments, the mSCF polypeptide comprises at least 4 contiguous amino acids of SEQ ID NO: 29, such as 5 to 13 contiguous amino acids of SEQ ID NO: 29. In some embodiments, the mSCF polypeptide is selected from any one of SEQ ID NOs: 51-57. In some embodiments, the mSCF polypeptide consists of the amino acid sequence according to SEQ ID NO: 54, 55, or 57. In some embodiments, the mSCF polypeptide comprises one or more of a biotin group, a palmitoyl group, a norleucine, or an aspartic acid methyl ester. In some embodiments, the biotin group is joined to the mSCF polypeptide by a C11 linker.

In some embodiments, the mSCF polypeptide is joined to a cell-penetrating peptide, such as a cell-penetrating peptide comprising the sequence of SEQ ID NO: 6 or 7. In some embodiments, the mSCF polypeptide is joined to the cell-penetrating peptide by a linker sequence, such as a linker sequence comprising the sequence of SEQ ID NO: 8.

In some embodiments, the composition comprises the MSH polypeptide. In some embodiments, the MSH polypeptide is a β-MSH polypeptide or a γ-MSH polypeptide. In some embodiments, the composition comprises the Endothelin polypeptide. In some embodiments, the Endothelin polypeptide is an Endothelin-1 polypeptide or an Endothelin-3 polypeptide. In some embodiments, the composition comprises histamine. In some embodiments, the composition comprises two or more of the mSCF polypeptide, the MSH polypeptide, the Endothelin polypeptide, or histamine.

In some embodiments, the composition further comprises a delivery vehicle, wherein the delivery vehicle is effective to increase delivery of the mSCF polypeptide across the epidermis of a human subject. In some embodiments, the delivery vehicle comprises microneedles, liposomes, or a chemical penetration enhancer.

In one aspect, provided herein is a use of any of the compositions disclosed herein in the manufacture of a medicament. In some embodiments, the medicament is for use in treating a condition of a human subject. In some embodiments, the condition comprises melanocyte deficiency or dysfunction. In some embodiments, the condition comprises hair loss or hair whitening. In some embodiments, the medicament is formulated for topical delivery to the skin of a human subject.

In one aspect, provided herein is a polynucleotide encoding an mSCF polypeptide of the present disclosure. The mSCF polypeptide may be an mSCF polypeptide according to any of the various aspects and embodiments disclosed herein, including with respect to the various methods and compositions disclosed herein.

In another aspect, provided herein is a cell comprising a polynucleotide of the present disclosure. The mSCF polypeptide may be an mSCF polypeptide according to any of the various aspects and embodiments disclosed herein, including with respect to the various methods and compositions disclosed herein.

### BRIEF DESCRIPTION OF THE DRAWINGS

FIG. 1 provides illustrative schematics of native murine SCF protein and its structural components at various stages.

FIG. 2 shows illustrative immunoblot results for a murine SCF polypeptide (“SCF”), showing its ability to induce phosphorylation of p42/44 at different time points: 5 minutes (5′), 15 minutes (15′) and 30 minutes (30′), compared to Vehicle (“Vhcl”).

FIG. 3 shows an illustrative schematic representation of an Mnt-1 cell-based in vitro platform for screening molecules and peptides of interest using activity (p42/44 phosphorylation) and/or functionality (apoptotic markers) analysis.

FIG. 4 shows illustrative immunoblot results for an assay comparing activities of human SCF (hSCF) and murine SCF (mSCF), as measured by phosphorylation of p42/44 in a human Mnt-1 cell line.

FIG. 5 shows illustrative immunoblot results for an activity assay of various mSCF polypeptides (indicated by SEQ ID NO, abbreviated “SID”), as measured by phosphorylation of p42/44 in a human Mnt-1 cell line.

FIG. 6 is a schematic diagram illustrating strategies for developing candidate mSCF polypeptides based on the native soluble mSCF.

FIG. 7 shows illustrative immunoblot results for an activity assay of various mSCF polypeptides (indicated by SEQ ID NO, abbreviated “SID”), as measured by phosphorylation of p42/44 in a human Mnt-1 cell line.

FIG. 8 is an immunoblot image showing an ability of an mSCF polypeptide according to SEQ ID NO: 20 (“SID: 20”) to reduce levels of the apoptotic marker cleaved caspase-3 (cCasp-3) at various concentrations (20 ng/ml, 200 ng/ml, and 500 ng/ml). “V” indicates addition of the indicated compound (i.e., wild-type mSCF (“SCF”), and/or ABT-199 (“ABT”)).

FIG. 9 shows illustrative immunoblot results for an anti-apoptosis activity assay of various mSCF polypeptides (indicated by SEQ ID NO, abbreviated “SID”), as measured by level of cCasp-3 or cleaved PARP (cPARP).

FIG. 10 shows illustrative immunoblot results for an activity assay of various mSCF polypeptides (indicated by SEQ ID NO, abbreviated “SID”), as measured by phosphorylation of p42/44 in a human Mnt-1 cell line.

FIG. 11 shows an immunoblot image analysis of non-SCF candidate molecules (α-MSH, β-MSH, γ-MSH, Endothelin 1 (Edn-1), and Histamine) and their ability to reduce cCasp-3 and cPARP levels in Mnt-1 cells. “V” indicates addition of ABT-199 (“ABT”).

FIG. 12 shows a sequence alignment of various SCF polypeptides according to SEQ ID NOs: 74, 1, 75, 20, 27, 29, and 37-43, from top to bottom, respectively.

FIG. 13 shows a sequence alignment of portions of human SCF (top; SEQ ID NO: 76) and murine SCF (bottom; SEQ ID NO: 77). The sequences appearing between SEQ ID NOs: 76 and 77 in the alignment represent those portions of SEQ ID NO: 76 that match corresponding positions of SEQ ID NO: 77. Positions of amino acid similarity are indicated by a “+”. The region corresponding to SEQ ID NO: 14 and its mutated variants (e.g., SEQ ID NOs: 20-36) is highlighted by a box.

FIG. 14 shows structural modelling illustrating structural features and binding interactions of human c-Kit receptor with human SCF (top) and an mSCF polypeptide (bottom).

FIG. 15 shows illustrative immunoblot results for an activity assay of various mSCF polypeptides (indicated by SEQ ID NO, abbreviated “SID”), as measured by phosphorylation of p42/44 in a human Mnt-1 cell line.

FIG. 16 shows illustrative immunoblot results for an anti-apoptosis activity assay of various mSCF polypeptides (indicated by SEQ ID NO, abbreviated “SID”), as measured by level of cCasp-3 or cleaved PARP (cPARP). “V” indicates addition of the ABT-199 (“ABT”).

FIG. 17 shows illustrative immunoblot results for an activity assay of various modified mSCF polypeptides (indicated by SEQ ID NO, abbreviated “SID”), as measured by phosphorylation of p42/44 in a human Mnt-1 cell line.

FIG. 18 shows illustrative imaging results for effects of treatment with a combination of mSCF polypeptides (“active molecules”) followed by ABT-199 treatment, as compared to vehicle in a human hair follicle explant model.

### DETAILED DESCRIPTION

The present disclosure provides murine stem cell factor (mSCF) polypeptides, melanocyte-stimulating hormone (MSH) polypeptides, and Endothelin polypeptides, and variants or fragments thereof, compositions comprising the same, and methods of using the same, including for treating a condition of a human subject.

In some embodiments, conditions to be treated include, but are not limited to, conditions associated with melanocyte deficiency or dysfunction, such as hair loss, hair whitening, and skin hypopigmentation. In some embodiments, mSCF polypeptides are used as cosmetic and/or therapeutic agents in preserving melanocyte stem cell function and preventing apoptosis of melanocytes, particularly in the context of hair pigmentation and skin regeneration.

### Definitions

As used herein, the term “fragment,” when referring to a protein or nucleic acid, for example, means any shorter sequence than a reference full-length protein or nucleic acid. Accordingly, any sequence of a nucleic acid or protein other than the full-length nucleic acid or protein sequence can be a fragment thereof. In some aspects, a protein fragment includes a fragment of a murine SCF polypeptide (“mSCF”), such as the mSCF polypeptide according to SEQ ID NO: 1, or a sequence variant thereof. Murine SCF is distinguishable from human SCF by sequence comparison. Variants of mSCF according to SEQ ID NO: 1 are distinguishable from human SCF by virtue of having greater sequence identity with mSCF than with human SCF, when optimally aligned.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, and mRNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example. As used herein, the terms “nucleic acid” and “nucleic acid molecule” can be used interchangeably, unless context clearly indicates otherwise.

As used herein, the term “polynucleotide” refers to a nucleic acid sequence that includes at least two nucleotide monomers. The term “polynucleotide” can refer to polymers of DNA, RNA, nucleic acid analogues, or combinations of these. A “polynucleotide” can be double-stranded or single-stranded and can be of any size. A polynucleotide can be a separate nucleic acid molecule or be a part of a nucleic acid molecule. Accordingly, the term “polynucleotide” can refer to a nucleic acid molecule or to a region of a nucleic acid molecule.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide, and comparing these sequences to a second nucleotide or amino acid sequence. As used herein, the term “percent (%) sequence identity” or “percent (%) identity” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). In some embodiments, ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±10%, or ±5%, or even ±1% from the specified value, as such variations are appropriate for the disclosed methods or to perform the disclosed methods.

As used herein, the term “subject” refers to any individual or patient on which methods disclosed herein may be performed. The term “subject” can be used interchangeably with the term “individual” or “patient.” The subject can be a human, although the subject may be a non-human animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment, including but not limited to a cosmetic improvement. For prophylactic benefit, the compositions may be administered to a subject at risk of developing a particular disease, condition, or symptom, or to a subject reporting one or more of the physiological symptoms of a disease or condition, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject), including but not limited to a prophylactic cosmetic effect.

The term “effective amount” or “therapeutically effective amount” refers to the amount of an agent that is sufficient to effect beneficial or desired results. The therapeutically effective amount may vary depending upon one or more of: the subject and disease/condition being treated, the weight and age of the subject, the severity of the disease/condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein.

As used herein, the terms “variant”, “functional variant” or “functional fragment” refer to a molecule, including a nucleic acid or protein, for example, that comprises a nucleotide and/or amino acid sequence that is altered by one or more nucleotides and/or amino acids compared to the nucleotide and/or amino acid sequences of the parent or reference molecule. For a protein, a functional variant is still able to function in a manner that is similar to the parent molecule. In other words, the modifications in the amino acid and/or nucleotide sequence of the parent molecule do not significantly affect or alter the functional characteristics of the molecule encoded by the nucleotide sequence or containing the amino acid sequence. The variant or functional variant may have conservative sequence modifications, such as nucleotide or amino acid substitutions. These modifications can be introduced by standard techniques known in the art, such as site-directed mutagenesis, or during chemical synthesis. Functional variants can also include, but are not limited to, derivatives that are substantially similar in primary structural sequence, but which contain, e.g., in vitro or in vivo modifications, chemical and/or biochemical, that are not found in the parent molecule. Non-limiting examples of such modifications include, inter alia, acetylation, acylation, ADP-ribosylation, amidation, covalent attachment of a lipid or lipid derivative, covalent attachment of phosphotidylinositol, cross-linking, cyclization, disulfide bond formation, demethylation, formation of covalent crosslinks, formation of cysteine, formation of pyroglutamate, formylation, gamma-carboxylation, glycosylation, hydroxylation, iodination, methylation, myristoylation, oxidation, pegylation, proteolytic processing, phosphorylation, prenylation, selenoylation, sulfation, and the like. In some embodiments, the modification is a biotinylation (e.g., an N-terminal biotinylation), and/or a palmitoylation (e.g., an N-terminal palmitoylation). In some embodiments, an mSCF polypeptide herein includes one or more modifications (e.g., two or more modifications, which may be the same or different). In some embodiments, a chemical modifying group (e.g., a biotin group) is joined to the polypeptide by a linker (e.g., a C11 linker).

As used herein, the term “conservative substitution” with respect to polypeptide variants refers to the replacement of one amino acid for another such that the replacement takes place within a family of amino acids that are related in their side chains. Genetically encoded amino acids can be divided into four families: (1) acidic (negatively charged), including Asp (D), Glu (G); (2) basic (positively charged), including Lys (K), Arg (R), His (H); (3) non-polar (hydrophobic), including Cys (C), Ala (A), Val (V), Leu (L), Ile (I), Pro (P), Phe (F), Met (M), Trp (W), Gly (G), Tyr (Y), with non-polar optionally being further subdivided into: (i) strongly hydrophobic (including Ala (A), Val (V), Leu (L), Ile (I), Met (M), Phe (F)), and (ii) moderately hydrophobic (including Gly (G), Pro (P), Cys (C), Tyr (Y), Trp (W)); and (4) uncharged polar, including Asn (N), Gln (Q), Ser(S), Thr (T). In some embodiments, a conservative substitution is a substitution with an analogue of a given amino acid, or a conservatively substituted amino acid thereof. For example, in some embodiments, a methionine is replaced with a leucine or a norleucine (Nle). Example mSCF polypeptides comprising an Nle substitution include, but are not limited to, those represented by SEQ ID NOs: 58 and 59. In some embodiments, one or more (e.g., 2 or more) aspartic acids are replaced with an aspartic acid methyl ester, such as Asp beta-methyl ester, designated “D(OMe)”. For example, one or both of the aspartic acids in an mSCF polypeptide comprising SEQ ID NO: 54 are substituted with D(OMe). In some embodiments, an mSCF polypeptide herein includes one or more conservative substitutions (e.g., two or more substitutions, which may be the same or different). In some embodiments, an mSCF polypeptide herein includes one or more conservative substitution (e.g., one or more D(OMe)) and one or more modification (e.g., a biotinylation).

### Illustrative Methods

In some aspects, provided herein are methods of treating a condition of a human subject. In some embodiments, the method comprises administering to the human subject a composition comprising one or more of a murine stem cell factor (mSCF) polypeptide, a melanocyte-stimulating hormone (MSH) polypeptide, or an Endothelin polypeptide. In some embodiments, the method comprises administering to the human subject a composition comprising an mSCF polypeptide. In some embodiments, the condition comprises melanocyte deficiency or dysfunction.

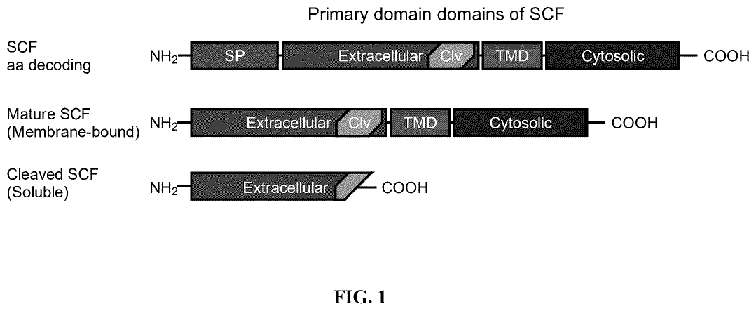

In some embodiments of the methods herein, the mSCF polypeptide is a soluble portion of a murine SCF, a variant thereof (comprising one or more amino acid substitutions, insertions, or deletions), or a functional fragment of any of these. Functionality may be determined by a suitable assay, such as an assay described herein, including but not limited to an assay that measures p42/44 phosphorylation in Mnt-1 cells (with p42/44 phosphorylation above that for an untreated control indicating activity). An exemplary structural schematic of an mSCF polypeptide is shown in FIG. 1, including the full length native mSCF (top row), a mature form of mSCF (middle row), and a cleaved soluble mSCF portion (bottom row). In some embodiments, the mSCF polypeptide comprises a polypeptide of SEQ ID NO: 1 (KEICGNPVTDNVKDITKLVANLPNDYMITLNYVAGMDVLPSHCWLRDMVIQLSLSLTT LLDKFSNISEGLSNYSIIDKLGKIVDDLVLCMEENAPKNIKESPKRPETRSFTPEEFFSIFNR SIDAFKDFMVASDTSDCVLSSTLGPEKDSRVSVTKPFMLPPVA), or a fragment or variant thereof.

In some embodiments, the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2 (KEICGNPVTDNVKDITKLVANLPNDYMITLNYVAGMDVLPSHCWLRDMVI). In some embodiments, the mSCF polypeptide comprises an N-terminal truncation of at least 10, at least 15, at least 20, at least 25, at least 30, at least 35, at least 40, at least 50, at least 60, or at least 70 amino acids as compared to SEQ ID NO: 1. In general, an N-terminal truncation relative to SEQ ID NO: 1 may be identified by having a region of optimal alignment between a given mSCF polypeptide and SEQ ID NO: 1 as located downstream of the N-terminus of SEQ ID NO: 1. Typically, such a truncation will reduce the maximum identity to the N-terminal sequence represented as SEQ ID NO: 2, such that the mSCF polypeptide does not comprise a stretch of 50 contiguous amino acids with more than 80%, more than 75%, more than 70%, more than 60%, or more than 50% sequence identity to SEQ ID NO: 2. In some embodiments, the mSCF polypeptide does not comprise 50 contiguous amino acids with more than 50% sequence identity to SEQ ID NO: 2. In further embodiments, the mSCF polypeptide does not comprise SEQ ID NO: 2.

In some embodiments, the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3 (FFSIFNRSIDAFKDFMVASDTSDCVLSSTLGPEKDSRVSVTKPFMLPPVA). In some embodiments, the mSCF polypeptide comprises a C-terminal truncation of at least 10, at least 15, at least 20, at least 25, at least 30, at least 35, at least 40, at least 45, or at least 50 amino acids as compared to SEQ ID NO: 1. In general, an C-terminal truncation relative to SEQ ID NO: 1 may be identified by having a region of optimal alignment between a given mSCF polypeptide and SEQ ID NO: 1 as located upstream of the C-terminus of SEQ ID NO: 1. Typically, such a truncation will reduce the maximum identity to the C-terminal sequence represented as SEQ ID NO: 3, such that the mSCF polypeptide does not comprise a stretch of 50 contiguous amino acids with more than 80%, more than 75%, more than 70%, more than 60%, or more than 50% sequence identity to SEQ ID NO: 3. In some embodiments, the mSCF polypeptide does not comprise 50 contiguous amino acids with more than 50% sequence identity to SEQ ID NO: 3. In further embodiments, the mSCF polypeptide does not comprise SEQ ID NO: 3. In some embodiments, the mSCF polypeptide comprises a C-terminal truncation in addition to an N-terminal truncation, relative to SEQ ID NO: 1. A truncation does not require that a given therapeutic protein have an mSCF-derived sequence at one or both termini. For example, an mSCF polypeptide comprising a truncation at one or both ends relative to SEQ ID NO: 1 may be incorporated into a fusion protein by fusion to one or more additional polypeptides (e.g., a cell penetrating peptide and optional linker). Note that the term “fusion” is not used to imply any particular method of production, and instead only indicates two or more sequence domains within a polypeptide chain. A variety of suitable methods for producing polypeptides described herein (including, but not limited to, mSCF polypeptides and fusion proteins) are available, non-limiting examples of which include chemical synthesis from component monomers, conjugation of component polypeptide chains, and expression of the entire fusion protein from a single transcript in cell-based gene expression systems (e.g., CHO cells). In some embodiments, a cell (e.g., a CHO cell) for the expression of a polypeptide described herein is modified to comprise a polynucleotide (e.g., DNA (genomically integrated or non-integrated), or RNA) encoding the polypeptide.

In some embodiments, a polypeptide according to the present disclosure (e.g., an mSCF polypeptide) comprises one or more modified amino acids. A polypeptide comprising a modified amino acid may include a modified amino acid that is incorporated during synthesis of the polypeptide (e.g., during a chain extension step of chemical synthesis), and/or an amino acid that is modified after synthesis of a precursor polypeptide (e.g., by chemical reaction with a terminal amino group, a terminal carboxy group, and/or one or more sidechains). In some embodiments, the modification is a biotinylation (e.g., an N-terminal biotinylation), and/or a palmitoylation (e.g., an N-terminal palmitoylation). In some embodiments, an mSCF polypeptide herein includes one or more modifications (e.g., two or more modifications, which may be the same or different). In some embodiments, a chemical modifying group (e.g., a biotin group) is joined to the polypeptide by a linker (e.g., a C11 linker). In some embodiments, an mSCF polypeptides comprises one or more Nle (e.g., an mSCF comprising SEQ ID NOs: 58 or 59). In some embodiments, one or more (e.g., 2 or more) aspartic acids are replaced with an aspartic acid methyl ester, such as Asp beta-methyl ester, designated “D(OMe)”. For example, one or both of the aspartic acids in an mSCF polypeptide comprising SEQ ID NO: 54 are substituted with D(OMe). In some embodiments, an mSCF polypeptide herein includes one or more conservative substitutions (e.g., two or more substitutions, which may be the same or different). In some embodiments, an mSCF polypeptide herein includes one or more conservative substitution (e.g., one or more D(OMe)) and one or more modification (e.g., a biotinylation).

In some embodiments, the mSCF polypeptide comprises a sequence with at least 80% identity (e.g., at least 85%, 90%, 95%, or 100% identity) to at least 10 contiguous amino acids (e.g., at least 20, 25, 30, 35, 40, 45, 50, 60, 75, 100, or 150 contiguous amino acids) of SEQ ID NO: 1. In some embodiments, the sequence identity is measured over at least 20 contiguous amino acids of SEQ ID NO: 1. In some embodiments, the identity is measured over at least 30 contiguous amino acids of SEQ ID NO: 1. In some embodiments, the at least 10 contiguous amino acids are located within positions 75 to 104 of SEQ ID NO: 1 (inclusive). In some embodiments, the percent identity is at least 90% identity over the sequence of contiguous amino acids of SEQ ID NO: 1. In some embodiments, the percent identity is 100% identity over the sequence of contiguous amino acids of SEQ ID NO: 1.

In some embodiments, the mSCF polypeptide comprises a sequence with at least 80%, at least 85%, at least 90%, at least 95%, or 100% identity to at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 100, at least 115, at least 120, at least 125, at least 130, at least 135, at least 140, at least 145, or at least 150 contiguous amino acids of SEQ ID NO: 1. In some embodiments, the mSCF polypeptide comprises a sequence with at least 90%, at least 91%, at least 92%, at least 93%, at least 94%, at least 95%, at least 96%, at least 97%, at least 98%, or at least 99% identity to at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 100, at least 115, at least 120, at least 125, at least 130, at least 135, at least 140, at least 145, or at least 150 contiguous amino acids of SEQ ID NO: 1. In some embodiments, the mSCF polypeptide comprises a sequence with at least 90%, at least 91%, at least 92%, at least 93%, at least 94%, at least 95%, at least 96%, at least 97%, at least 98%, or at least 99% identity to SEQ ID NO: 1. In some embodiments, the mSCF comprises the sequence of SEQ ID NO: 1.

In some embodiments, the mSCF polypeptide is a variant of the mSCF polypeptide according to SEQ ID NO: 1, such that it includes one or more amino acid substitutions, insertions, and/or deletions relative to SEQ ID NO: 1. For example, in some embodiments, the mSCF polypeptide comprises a sequence with between 80% to 99% identity to at least 20 contiguous amino acids of SEQ ID NO: 1. In some embodiments, the mSCF polypeptide comprises one or more amino acid substitutions as compared to said at least 20 contiguous amino acids of SEQ ID NO: 1. In some embodiments, the mSCF polypeptide comprises (i) a sequence with between 80% to 99% identity to at least 10 contiguous amino acids (e.g., at least 20, 25, 30, 35, 40, 45, 50, 60, 75, 100, or 150 contiguous amino acids) of SEQ ID NO: 1, and (ii) one or more amino acid substitutions as compared to said at least 20 contiguous amino acids (e.g., e.g., at least 20, 25, 30, 35, 40, 45, 50, 60, 75, 100, or 150 contiguous amino acids) of SEQ ID NO: 1. In some embodiments, the sequence identity is measured over at least 20 contiguous amino acids of SEQ ID NO: 1. In some embodiments, the identity is measured over at least 30 contiguous amino acids of SEQ ID NO: 1. In some embodiments, the at least 10 contiguous amino acids are located within positions 75 to 104 of SEQ ID NO: 1 (inclusive). In some embodiments, the percent identity is at least 90% identity over the sequence of contiguous amino acids of SEQ ID NO: 1. In some embodiments, the percent identity is at least 95% identity over the sequence of contiguous amino acids of SEQ ID NO: 1.

In some embodiments, the mSCF polypeptide comprises (i) a sequence with between 80% to 99% identity to at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 100, at least 115, at least 120, at least 125, at least 130, at least 135, at least 140, at least 145, or at least 150 contiguous amino acids of SEQ ID NO: 1, and (ii) one or more amino acid substitutions as compared to said at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 100, at least 115, at least 120, at least 125, at least 130, at least 135, at least 140, at least 145, or at least 150 contiguous amino acids of SEQ ID NO: 1. In some embodiments, the mSCF polypeptide comprises (i) a sequence with between 90% to 99% identity to at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 100, at least 115, at least 120, at least 125, at least 130, at least 135, at least 140, at least 145, or at least 150 contiguous amino acids of SEQ ID NO: 1, and (ii) one or more amino acid substitutions as compared to said at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 100, at least 115, at least 120, at least 125, at least 130, at least 135, at least 140, at least 145, or at least 150 contiguous amino acids of SEQ ID NO: 1.

In some embodiments, the mSCF polypeptide comprises one or more amino acid substitutions relative to SEQ ID NO: 1. In some embodiments, the one or more substitutions comprise a substation of the amino acid at one or more of positions corresponding to one or more of position 77, 78, 81, 83, 85, 87, 88, 89, 92, 93, 94, 95, 97, 98, 99, 100, 101, 102, 103, or 104 of SEQ ID NO: 1. In some embodiments, the one or more amino acid substitutions include one or more of D77H, K78E, K78D, K81I, V83E, D85Y, D85E, D85F, V87F, V87W, L88F, C89D, E92D, N93E, N93Q, N93D, A94P, P95D, P95K, P95V, N97I, N97K, 198K, 198D, 198N, 198E, K99D, K99E, K99I, K99W, K99T, K99Y, E100F, E100V, S101F, S101Y, S101D, S101E, P102K, P102F, P102Y, K103R, K103P, or R104Y, relative to SEQ ID NO: 1. Amino acid substitutions (also referred to as “mutations”) are indicated by the form “AxxB,” relative to a reference sequence (e.g., SEQ ID NO: 1), where “B” is the amino acid that replaces amino acid “A” at position “xx” of the reference sequence. In cases where a variant polypeptide with one or more specified mutations and the reference sequence are not otherwise 100% identical to one another, the numerical position of one or more of the amino acid substitutions indicated for the corresponding variant may shift (e.g., in the case of insertion or deletion of one or more amino acids relative to the reference sequence), but still be contained within the resulting variant. For example, the histidine substitution corresponding to “D77H” (at position 77 relative to SEQ ID NO: 1) may be present, but at a different position in the resulting variant (e.g., at position 3 of a polypeptide according to SEQ ID NO: 20). Whether an amino acid corresponds to an indicated substitution, albeit at a different position, can be determined by sequence alignment. In general, an alignment showing identity or similarity of one or more amino acids flanking the amino acid substitution will allow the variant amino acid in a homologous query sequence to be positioned locally with respect to the corresponding variant of a reference sequence to confirm the presence of the substitution, albeit at a shifted numerical position in a given polypeptide chain. In some embodiments, a region comprising at least three to fifteen amino acids, including the substituted position, will locally align with the corresponding variant of the reference sequence with a relatively high percent identity or similarity, including at the position of the substituted amino acid along the corresponding variant sequence (e.g., 90%, 95%, or 100% identity). In some embodiments, the one or more amino acid substitutions (e.g. all or less than all of the amino acid substitutions) indicated by a corresponding variant of the reference sequence is considered to be present in a given variant, even if occurring at a different physical position along a polypeptide chain, if the sequence of the polypeptide being compared aligns to the corresponding variant with an identical amino acid at the indicated position along the corresponding variant sequence when using a BLASTP alignment algorithm with default parameters. Alignments can also be used to identify a position in a starting sequence that should be modified in order to arrive at a polypeptide corresponding to a variant polypeptide disclosed herein, including in cases where the position and/or identity of the starting amino acid to be modified is different from that of the corresponding variant. In general, two amino acids are considered to be similar if having chemical properties sufficiently similar for contributing to an alignment using default parameters. In some embodiments, a given amino acid variant is considered present when at least fifteen amino acids (including the indicated variant position(s)) aligns to the indicated numbered position of the reference sequence (modified to contain the indicated variant(s)) with 100% identity, using a BLASTP alignment algorithm with default parameters.

In some embodiments, the mSCF polypeptide comprises a sequence with at least 80%, at least 85%, at least 90%, at least 95%, or 100% identity to at least 20, at least 21, at least 22, at least 23, at least 24, at least 25, at least 26, at least 27, at least 28, at least 29, or 30 contiguous amino acids of SEQ ID NO: 4 (IIHKLGIIVDDLFFCMEENAPKNIKEFPKR). In some embodiments, the mSCF polypeptide comprises a sequence with at least 90%, at least 95%, or 100% identity to at least 20, at least 21, at least 22, at least 23, at least 24, at least 25, at least 26, at least 27, at least 28, at least 29, or 30 contiguous amino acids of SEQ ID NO: 4. In some embodiments, the mSCF polypeptide comprises a sequence with at least 90%, at least 91%, at least 92%, at least 93%, at least 94%, at least 95%, at least 96%, at least 97%, at least 98%, at least 99% or 100% identity to at least 20, at least 21, at least 22, at least 23, at least 24, at least 25, at least 26, at least 27, at least 28, at least 29, or 30 contiguous amino acids of SEQ ID NO: 4.

In some embodiments, the mSCF polypeptide comprises a sequence with at least 80%, at least 85%, at least 90%, at least 95%, or 100% identity to SEQ ID NO: 4. In some embodiments, the mSCF polypeptide comprises a sequence with at least 90%, at least 95%, or 100% identity to SEQ ID NO: 4. In some embodiments, the mSCF polypeptide comprises a sequence with at least 90%, at least 91%, at least 92%, at least 93%, at least 94%, at least 95%, at least 96%, at least 97%, at least 98%, at least 99% or 100% identity to SEQ ID NO: 4. In some embodiments, the mSCF polypeptide comprises the amino acid sequence of SEQ ID NO: 4.

In some embodiments, the mSCF polypeptide comprises a sequence with at least 80%, at least 85%, at least 90%, at least 95%, or 100% identity to SEQ ID NO: 5 (IIHX₁LGIIX₂DX₃LX₄FX₅MEX₆X₇X₈X₉KX₁₀X₁₁X₁₂X₁₃X₁₄X₁₅X₁₆, where X₁ is E, D, or K; X₂ is V or E; X₃ is D, Y, E, or F; X₄ is F or W; X₅ is C or D; X₆ is E or D; X₇ is N, E, Q, or D; X₈ is A or P; X₉ is P, D, K, or V; X₁₀ is N, I, or K; X₁₁ is I, K, D, N, or E; X₁₂ is K, D, E, I, W, T, or Y; X₁₃ is E, F, or V; X₁₄ is F, Y, D, or E; X₁₅ is P, K, F, or Y; X₁₆ is K, R, or P). In some embodiments, the mSCF polypeptide comprises a sequence with at least 90% identity to SEQ ID NO: 5. In some embodiments, the mSCF polypeptide comprises the amino acid sequence of SEQ ID NO: 5.

Illustrative SCF polypeptides are set forth in Table 1 below.

TABLE 1
Illustrative SCF polypeptides

| Sequence | SEQ ID NO |
|---|---|
| MKEICGNPVTDNVKDITKLVANLPNDYMIT | 9 |
| ITKLVANLPNDYMITLNYVAGMDVLPSHCW | 10 |
| LNYVAGMDVLPSHCWLRDMVIQLSLSLTTL | 11 |
| LRDMVIQLSLSLTTLLDKFSNISEGLSNYS | 12 |
| LDKFSNISEGLSNYSIIDKLGKIVDDLVLC | 13 |
| IIDKLGKIVDDLVLCMEENAPKNIKESPKR | 14 |
| MEENAPKNIKESPKRPETRSFTPEEFFSIF | 15 |
| PETRSFTPEEFFSIFNRSIDAFKDFMVASD | 16 |
| NRSIDAFKDFMVASDTSDCVLSSTLGPEKD | 17 |
| TSDCVLSSTLGPEKDSRVSVTKPFMLPPV | 18 |
| LDKASNISEGLSNYSIIHKLGIIVDDLFFC | 19 |
| IIHKLGIIVDDLFFCMEENAPKNIKEFPKR | 20 |
| IIHELGIIVDDLFFCMEENADKNIDEFPKR | 21 |
| IIHKLGIIVDYLFFCMEENAPKNIKEYPK | 22 |
| IIHKLGIIVDELFFCMEEEPKNIKEFDKR | 23 |
| IIHKLGIIVDDLFFCMEENAPKNDKEDPKR | 24 |
| IIHKLGIIVDDLFFCMEENAVPKNIVEFPKR | 25 |
| IIHDLGIIVDDLFFCMEENAPKNEKEFYKR | 26 |
| IIHKLGIIVDFLFFCMEENAPKNIWEFPKY | 27 |
| IIHKLGIIVDELFFCMEENAPKNIEEFPKR | 28 |
| IIHKLGIIVDDLFFCMEENAPKNITEFPKR | 29 |
| IIHKLGIIVDDLFFCMEEQAPKNIDEFPKR | 30 |
| IIHELGIIVDELFFCMEEDAPKNIEEFPKR | 31 |
| IIHDLGIIVDYLFFCMEEDAPKNIWEFPKR | 32 |
| IIHELGIIVDFLFFCMEEDAPKNIYEFPKR | 33 |
| IIHELGIIVDELFFCMEEQAPKNIEEFPKR | 34 |
| IIHELGIIVDELFFCMEENAPKNIDEFPKR | 35 |
| IIHELGIIEDDLWFDMEDNAPKNIEEFPKR | 36 |
| WISEMVVQLSDSLTDLLDKFSNISEGLSNYSIIDKLVNIVDDL | 37 |
| VECVKENS | |
| WLRDMVIQLSLSLTTLLDKFSNISEGLSNYSIIDKLGKIVDDL | 38 |
| VLCMEENA | |
| WISEMVVQLSDSLTDLLDKASNISEGLSNYSIIHKLVIIVDDLF | 39 |
| FCVKENS | |
| WLRDMVIQLSLSLTTLLDKASNISEGLSNYSIIHKLGIIVDDLF | 40 |
| FCMEENA | |
| WISEMVVQLSDSLTDLLDKASNISEGLSNYSIIHKLVNIVDDL | 41 |
| FECVKENS | |
| WISEMVVQLSDSLTDLLDKFSNISEGLSNYSIIDKLVIIVDDLV | 42 |
| FCVKENS | |
| WISEMVVQISLSLTTLLDKFSNISEGLSNYSIIDKLVKIVDDLV | 43 |
| LCVKENS | |
| KEICGNPVTDNVKDITKLVANLPNDYMITLNYVAGMDVLPS | 44 |
| HCWLRDMVIQLSLSLTTLLDKFSNISEGLSNYSIIDKLGKIVD | |
| DLVLCMEENAPKNIKESPKRPETRSFTPEEFFSIFNRSIDAFKD | |
| FMVASDTSDCVLSSTLGPEKDSRVSVTKPFMLPPVA | |
| KEICGAPVTDNVKDITKLVANLPNDYMITLNYVAGMDVLPS | 45 |
| HCWLRDMVIQLSLSLTTLLDKASNISEGLSNYSIIHKLGIIVDD | |
| LFFCMEENAPKNIKEFPKRPETRSFTPEEFFSIFNRSIDAFKDF | |
| MVASDTSDCVLSSTLGPEKDSRVSVTKPFMLPPVA | |
| MKEICGNPVTDNVKDITKLVANLPNDYMITLNYVAGMDVLP | 46 |
| SHCWLRDMVIQLSLSLTTLLDKFSNISEGLSNYSIIDKLGKIVD | |
| DLVLCMEENAPKNIKESPKRPETRSFTPEEFFSIFNRSIDAFKD | |
| FMVASDTSDCVLSSTLGPEKDSRVSVTKPFMLPPVA | |
| MKEICGAPVTDNVKDITKLVANLPNDYMITLNYVAGMDVLP | 47 |
| SHCWLRDMVIQLSLSLTTLLDKASNISEGLSNYSIIHKLGIIVD | |
| DLFFCMEENAPKNIKEFPKRPETRSFTPEEFFSIFNRSIDAFKD | |
| FMVASDTSDCVLSSTLGPEKDSRVSVTKPFMLPPVA | |
| MDRTTLTNGGGGSKEICGNPVTDNVKDITKLVANLPNDYMI | 48 |
| TLNYVAGMDVLPSHCWLRDMVIQLSLSLTTLLDKFSNISEGL | |
| SNYSIIDKLGKIVDDLVLCMEENAPKNIKESPKRPETRSFTPEE | |
| FFSIFNRSIDAFKDFMVASDTSDCVLSSTLGPEKDSRVSVTKP | |
| FMLPPVA | |
| MRRRRRRRGGGGSKEICGNPVTDNVKDITKLVANLPNDYM | 49 |
| ITLNYVAGMDVLPSHCWLRDMVIQLSLSLTTLLDKFSNISEG | |
| LSNYSIIDKLGKIVDDLVLCMEENAPKNIKESPKRPETRSFTPE | |
| EFFSIFNRSIDAFKDFMVASDTSDCVLSSTLGPEKDSRVSVTK | |
| PFMLPPVA | |
| MDRTTLTNGGGGSKEICGAPVTDNVKDITKLVANLPNDYMI | 50 |
| TLNYVAGMDVLPSHCWLRDMVIQLSLSLTTLLDKASNISEGL | |
| SNYSIIHKLGIIVDDLFFCMEENAPKNIKEFPKRPETRSFTPEEF | |
| FSIFNRSIDAFKDFMVASDTSDCVLSSTLGPEKDSRVSVTKPF | |
| MLPPVA | |
| ENAPKNITEFPKR | 51 |
| PKNITEFPKR | 52 |
| IHKLG | 53 |
| IIVDD | 54 |
| DDLFF | 55 |
| KNITE | 56 |
| NITEF | 57 |
| IIHKLGIIVDDLFFCXEENAPKNIKEFPKR (where X = Nle) | 58 |
| IIHKLGIIVDDLFFCXEENAPKNITEFPKR (where X = Nle) | 59 |
| Biotin-IHKLG | 60 |
| Biotin-IIVDD | 61 |
| Biotin-DDLFF | 62 |
| Biotin-IIVD*D (where D* = D(OMe)) | 63 |
| Biotin-IIVDD* (where D* = D(OMe)) | 64 |
| Biotin-IIVD*D* (where D* = D(OMe)) | 65 |
| Palmitoyl-IIHKLGIIVDDLFFCXEENAPKNIKEFPKR | 66 |
| (where X = Nle) | |
| Palmitoyl-IIHKLGIIVDDLFFCXEENAPKNITEFPKR | 67 |
| (where X = Nle) | |
| Palmitoyl-IHKLG | 68 |
| Palmitoyl-IIVDD | 69 |
| Palmitoyl-DDLFF | 70 |
| Biotin-C11-IHKLG | 71 |
| Biotin-C11-IIVDD | 72 |
| Biotin-C11-DDLFF | 73 |

In some embodiments, the mSCF polypeptide comprises a sequence with at least 80%, at least 85%, at least 90%, or at least 95% sequence identity to any one of SEQ ID NOs: 15-36, 44, 58, or 59. In some embodiments, the mSCF polypeptide comprises a sequence with at least 90% sequence identity to any one of SEQ ID NOs: 15-36, 44, 58, or 59. In some embodiments, the mSCF polypeptide comprises a sequence with at least 95% sequence identity to any one of SEQ ID NOs: 15-36, 44, 58, or 59. In some embodiments, the mSCF polypeptide comprises the sequence of any one of SEQ ID NOs: 15-36, 44, 58, or 59. In some embodiments, the mSCF polypeptide consists of the sequence of any one of SEQ ID NOs: 15-36, 44, 58, or 59.

In some embodiments, the mSCF polypeptide comprises the sequence of SEQ ID NO: 15, or a variant thereof. In some embodiments, the mSCF polypeptide comprises the sequence of SEQ ID NO: 16, or a variant thereof. In some embodiments, the mSCF polypeptide comprises the sequence of SEQ ID NO: 17, or a variant thereof. In some embodiments, the mSCF polypeptide comprises the sequence of SEQ ID NO: 18, or a variant thereof. In some embodiments, the mSCF polypeptide comprises the sequence of SEQ ID NO: 19, or a variant thereof. In some embodiments, the mSCF polypeptide comprises the sequence of SEQ ID NO: 20, or a variant thereof. In some embodiments, the mSCF polypeptide comprises the sequence of SEQ ID NO: 21, or a variant thereof. In some embodiments, the mSCF polypeptide comprises the sequence of SEQ ID NO: 22, or a variant thereof. In some embodiments, the mSCF polypeptide comprises the sequence of SEQ ID NO: 23, or a variant thereof. In some embodiments, the mSCF polypeptide comprises the sequence of SEQ ID NO: 24, or a variant thereof. In some embodiments, the mSCF polypeptide comprises the sequence of SEQ ID NO: 25, or a variant thereof. In some embodiments, the mSCF polypeptide comprises the sequence of SEQ ID NO: 26, or a variant thereof. In some embodiments, the mSCF polypeptide comprises the sequence of SEQ ID NO: 27, or a variant thereof. In some embodiments, the mSCF polypeptide comprises the sequence of SEQ ID NO: 28, or a variant thereof. In some embodiments, the mSCF polypeptide comprises the sequence of SEQ ID NO: 29, or a variant thereof. In some embodiments, the mSCF polypeptide comprises the sequence of SEQ ID NO: 30, or a variant thereof. In some embodiments, the mSCF polypeptide comprises the sequence of SEQ ID NO: 31, or a variant thereof. In some embodiments, the mSCF polypeptide comprises the sequence of SEQ ID NO: 32, or a variant thereof. In some embodiments, the mSCF polypeptide comprises the sequence of SEQ ID NO: 33, or a variant thereof. In some embodiments, the mSCF polypeptide comprises the sequence of SEQ ID NO: 34, or a variant thereof. In some embodiments, the mSCF polypeptide comprises the sequence of SEQ ID NO: 35, or a variant thereof. In some embodiments, the mSCF polypeptide comprises the sequence of SEQ ID NO: 36, or a variant thereof. In some embodiments, the mSCF polypeptide comprises the sequence of SEQ ID NO: 44, or a variant thereof. In some embodiments, the mSCF polypeptide comprises the sequence of SEQ ID NO: 58, or a variant thereof. In some embodiments, the mSCF polypeptide comprises the sequence of SEQ ID NO: 59, or a variant thereof.

In some embodiments, the mSCF polypeptide comprises a sequence with at least 80%, at least 85%, at least 90%, or at least 95% sequence identity to any one of SEQ ID NOs: 78-167. In some embodiments, the mSCF polypeptide comprises a sequence with at least 90% sequence identity to any one of SEQ ID NOs: 78-167. In some embodiments, the mSCF polypeptide comprises a sequence with at least 95% sequence identity to any one of SEQ ID NOs: 78-167. In some embodiments, the mSCF polypeptide comprises the sequence of any one of SEQ ID NOs: 78-167. In some embodiments, the mSCF polypeptide consists of the sequence of any one of SEQ ID NOs: 78-167.

The mSCF polypeptide may be of a variety of lengths. In some embodiments, the mSCF polypeptide is at least 5, at least 10, at least 15, 20, at least 25, at least 30, at least 35, at least 40, at least 45, at least 50, at least 60, at least 75, at least 100, or at least 150 amino acids in length. In some embodiments, the mSCF polypeptide is at least 20 amino acids in length. In some embodiments, the mSCF polypeptide is at least 30 amino acids in length. In some embodiments, the mSCF polypeptide is 5 amino acids to 150 amino acids in length. In some embodiments, the mSCF polypeptide is 20 amino acids to 75 amino acids in length. In some embodiments, the mSCF polypeptide is 30 to 60 amino acids in length. In some embodiments, the mSCF polypeptide is 40 to 50 amino acids in length. In some embodiments, the mSCF polypeptide is about 30 amino acids in length.

In some embodiments, the mSCF polypeptide comprises at least 4, at least 5, at least 6, at least 7, at least 8, at least 9, at least 10, at least 15, at least 20, at least 25, or 30 contiguous amino acids of SEQ ID NO: 29 (IIHKLGIIVDDLFFCMEENAPKNITEFPKR). In some embodiments, the mSCF polypeptide comprises 5 to 13 contiguous amino acids of SEQ ID NO: 29. In some embodiments, the mSCF polypeptide comprises 6 to 12 contiguous amino acids of SEQ ID NO: 29. In some embodiments, the mSCF polypeptide comprises 7 to 11 contiguous amino acids of SEQ ID NO: 29. In some embodiments, the mSCF polypeptide comprises 8 to 10 contiguous amino acids of SEQ ID NO: 29. In some embodiments, the mSCF polypeptide further comprises at least 80% sequence identity (e.g., at least 90%, or at least 95% identity) to SEQ ID NO: 29. In some embodiments, the mSCF polypeptide comprises the amino acid sequence of SEQ ID NO: 29.

In some embodiments, the mSCF polypeptide comprises one or more of SEQ ID NOs: 51-57. In some embodiments, the mSCF polypeptide comprises one or more of SEQ ID NOs: 54, 55, or 57. In some embodiments, the mSCF polypeptide is selected from any one of SEQ ID NOs: 51-57. In some embodiments, the mSCF polypeptide consists of the amino acid sequence according to SEQ ID NO: 54 (IIVDD), SEQ ID NO: 55 (DDLFF), or SEQ ID NO: 57 (NITEF).

In some embodiments, the mSCF polypeptide is joined to a cell-penetrating peptide. A variety of cell-penetrating peptides are available to those skilled in the art. In general, a cell penetrating peptide is a peptide which can facilitate transportation of an associated polypeptide from in vitro and/or in vivo to inside a cell, without causing significant membrane damage. In some embodiments, the cell-penetrating peptide comprises the amino acid sequence of SEQ ID NO: 6 (DRTTLTN) or SEQ ID NO: 7 (RRRRRRR). Further non-limiting examples of cell-penetrating peptides are provided in US20150307859A1 and US20210070823A1, which are incorporated herein by reference. In some embodiments, one or more cell-penetrating polypeptides are located at the N-terminus of a protein comprising an mSCF polypeptide. In some embodiments, one or more cell-penetrating polypeptides are located at the C-terminus of a protein comprising an mSCF polypeptide. In some embodiments, one or more cell-penetrating polypeptides are located at both ends of a protein comprising an mSCF polypeptide.

In some embodiments, the mSCF polypeptide is joined to the cell-penetrating peptide by a linker sequence. As used herein, the term “linker sequence” refers to a sequence of amino acids within a polypeptide chain that connect different protein domains. Linkers may also act as spacers between the domains of multidomain proteins. Linkers are typically relatively short and relatively flexible polypeptides. A variety of suitable linkers are available to those skilled in the art. In some embodiments, the linker sequence comprises the amino acid sequence of SEQ ID NO: 8 (GGGGS), which may optionally be repeated one or more times.

In some embodiments, the method comprises administering a composition comprising an MSH polypeptide. Wild-type MSH activates melanocytes via the melanocortin 1 receptor (MC1R). This interaction stimulates melanin production, which helps maintain hair pigmentation and skin color. In some embodiments, polypeptides are designed to mimic or enhance MSH signaling to increase melanocyte cell activation, improve hair pigmentation, and/or aid in the regeneration of the skin and hair follicles. In some embodiments, the MSH polypeptide is a β-MSH polypeptide. In some embodiments, the MSH polypeptide is a γ-MSH polypeptide. In some embodiments, the method comprises administering an MSH polypeptide in combination with an mSCF polypeptide.

In some embodiments, the method comprises administering a composition comprising an Endothelin polypeptide. Wild-type endothelins are proteins that are produced by endothelial cells, which line blood vessels. Endothelins are members of a family of peptides that regulate blood pressure and other physiological processes in the body. In some embodiments, the Endothelin polypeptide is an Endothelin-1 (Edn-1) polypeptide. In some embodiments, the Endothelin polypeptide is an Endothelin-3 (Edn-3) polypeptide. In some embodiments, the method comprises administering an Endothelin polypeptide in combination with one or more of an mSCF polypeptide or an MSH polypeptide.

### Methods of Treatment

In various embodiments, methods provided herein are for the treatment of a condition in a human subject. In some embodiments, the condition to be treated by the methods herein comprises melanocyte deficiency or dysfunction. In general, deficiency or dysfunction may be determined with regard to a reference, such as a level of melanocytes or melanocyte function for a reference healthy tissue or subject, or for a reference time point for a particular subject experiencing such deficiency or dysfunction. In some embodiments, melanocyte deficiency or dysfunction is characterized by a loss or absence of melanocytes in certain areas of the skin of a subject (e.g., skin of the scalp or face), thereby resulting in a loss of pigmentation in the hair and/or skin of a subject. In other words, a subject having melanocyte deficiency or dysfunction may have a loss off hair, lighter colored hair, loss of skin color, or lighter skin color when compared to a reference (e.g., a reference healthy subject). In some embodiments, the condition to be treated comprises hair loss and/or hair whitening.

In one embodiment, the treating comprises one or more of (i) inhibiting apoptosis of melanocytes or melanocyte stem cells (McSCs), (ii) increasing number or survival of melanocytes or McSCs, (iii) increasing a level of melanin synthesis, (iv) inhibiting hair whitening, (v) promoting increased hair pigmentation, (vi) inhibiting hair loss, or (vii) promoting increased hair growth; as compared to the absence of said treating. In some embodiments, the treating comprises inhibiting apoptosis of melanocytes or McSCs, which can be characterized by said melanocytes or McSCs not undergoing cell death for a given period of time, or undergoing cell death at a reduced rate relative to a rate prior to treatment. In some embodiments, the treating comprises increasing number or survival of melanocytes or McSCs as compared to the absence of said treating. In some embodiments, the treating comprises increasing a level of melanin synthesis as compared to the absence of said treating. In some embodiments, the treating comprises inhibiting hair whitening as compared to the absence of said treating, such that rate of color loss in hair at a given location and/or the rate at which an area affected by loss of color spreads is reduced, as compared to the absence of said treating. In some embodiments, inhibition of hair whitening is characterized by the delay or halt of hair whitening progression, and processes involved with the same. In some embodiments, the treating comprises promoting increased hair pigmentation as compared to the absence of said treating. Such increased hair pigmentation can be characterized by an increase in hair color. In some embodiments, the treating comprises inhibiting hair loss as compared to the absence of said treating. In some embodiments, inhibiting hair loss is characterized by the absence of hair loss over a given period of time, or hair loss at a reduced rate as compared to a reference (e.g., a pre-treatment rate of hair loss). In some embodiments, the treating comprises promoting increased hair growth as compared to the absence of said treating. In some embodiments, the treating is effective to reduce a rate of hair whitening. In some embodiments, the treating is effective to promote an increase in hair pigmentation in the subject as compared to the absence of said treating.

In some embodiments, the administering comprises topical administration of the composition to the skin of the human subject. In some embodiments, the skin comprises the scalp or face of the human subject. A variety of suitable delivery approaches and delivery vehicles are available, which can be utilized and optionally tailored to optimize penetration, bioavailability, and targeted activity within the skin and hair follicle structures. For example, topical formulations, such as creams, gels, or serums, which offer a non-invasive and consumer-friendly application method, can be used. These formulations can utilize alcohols within the solution to enhance skin penetration, enabling deeper delivery of active molecules. Additionally, permeation enhancers such as polysorbate-20, propylene glycol, or dimethyl sulfoxide (DMSO) can be incorporated to temporarily disrupt the stratum corneum barrier, allowing better access to hair follicles and deeper skin layers. In some embodiments, nanoparticle-based delivery methods are used. Nanoparticle-based delivery offers a promising approach for facilitating targeted molecule delivery to hair follicles. Both encapsulation and surface display of active components can be utilized to protect the molecules from degradation, improve skin penetration, and enhance bioavailability within the follicular niche. Nanoparticles that are either biodegradable or inert can be used, facilitating safety and compatibility with human skin. Nanoparticles can be engineered for controlled release, allowing for, e.g., a sustained therapeutic effect within the hair follicle and surrounding areas. This approach is particularly advantageous for precise targeting of follicular structures, promoting melanocyte activation and survival. In some embodiments, microneedle-assisted delivery methods are used. Microneedling is a minimally invasive technique that enhances skin permeability by creating microchannels in the epidermis, allowing for deeper penetration of topically applied molecules. When combined with the compositions, polypeptides, proteins, compounds, and molecules herein, microneedling facilitates targeted delivery into the hair follicle, enabling direct access to melanocyte stem cells within the follicular niche. This method can be particularly beneficial for larger or charged molecules that might otherwise struggle to penetrate intact skin barriers. Additionally, microneedling may stimulate localized skin repair processes, which could synergize with the compositions, polypeptides, proteins, compounds, and molecules herein to further support melanocyte activation.

In some embodiments, the method comprises a delivery vehicle as set forth above. In some embodiments, the delivery vehicle is effective to increase delivery of the mSCF polypeptide across the epidermis of a human subject. In some embodiments, the delivery vehicle comprises microneedles, liposomes, or a chemical penetration enhancer. Lipid carrier vehicles (e.g., liposomes and lipid-derived nanoparticles) that can be used herein are generally useful in a variety of applications in research and medicine, particularly for their use as transfer vehicles of diagnostic or therapeutic compounds in vivo, and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains. Bilayer membranes of the liposomes can also be formed by amphiphilic polymers and surfactants. In some embodiments, lipid-based delivery vehicles include lipid nanoparticles. In contrast to liposomes and cationic liposomes, lipid nanoparticles (LNPs) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. Lipid nanoparticle can have a diameter in the range of from 10 nm to 1000 nm (more commonly, smaller than 120 nm or even 100 nm). A variety of lipid nanoparticles suitable for delivery of compositions to the skin of the subject are available to the skilled person.

Likewise, a variety of cell penetration enhancers for the delivery of compounds to the skin of a subject are available to those skilled in the art. Non-limiting examples of cell penetration enhancers include, for instance, ethanol, acetone, detergents, bases, propylene glycol, pyrriolidones, dimethylacetamide, dimethylformamide, dimethylsulfoxide, alkyl sulfoxide, phosphine oxide, surfactants and caprolactams such as azone. Other compounds that may be used for penetration enhancement include amines and amides, alkyl N,N-distributed-amino acetates, decylmethylsulfoxide, pyrrolidones, pirotiodecane (HPE-101), benzlyalkonium, benzylalkonium chloride polymers, silicone based polymers, fatty acids, cyclic ureas, terpenes, liposomes, and cyclodextrins. Further non-limiting examples of cell penetration enhancers are described in for instance, in US20080233142A1, which are incorporated herein by reference.

In some embodiments, compositions for use in the present methods are delivered as part of a cosmetic product. Examples of cosmetic products include, without limitation, personal care products, skin products, skin creams, skin gels, skin ointments, skin lotions, hair and skin conditioners, moisturizers, skin patches, skin masks, tissue wipes, sunscreens, sunblocks, mousse, sprays, styling gels, bath and shower gels, shampoos, conditioners, cream rinses, hair sprays, hair dyes and coloring products, soaps, body scrubs, exfoliants, astringents, leave-on conditioners, cleansers, or the like; whether in the form of creams, lotions, gels, ointments, macro-emulsions, micro-emulsions, nano-emulsions, serums, balms, colloids, solutions, liquids, suspensions, dispersions, compacts, solids, powders, spray-on formulations, brush-on formulations, patches, microneedle patches, skin delivery enhancing systems, bandage, tissue cloths, wipes, masks, aerosols, pastes, soap bars, cosmetic devices, and/or any other suitable forms known to those skilled in the art.

The compositions for use in methods disclosed herein can also contain one or more pharmaceutically acceptable carriers and/or excipients acceptable for a mode of administration (e.g., for topical application and/or for subcutaneous administration). The carrier and/or excipient can be in any of a variety of forms. Non-limiting examples of suitable carriers and/or excipients include simple solutions (e.g., water or oil based), emulsions, dispersions, multi-phase systems, semi-solid forms, solid forms (powder, sticks, patches), skin masks, tissues, foams, and aerosols. For example, emulsion carriers and/or excipients can include, but are not limited to, oil-in-water, silicone-in-water, water-in-oil, water-in-silicone, water-in-oil-in-water, oil-in-water-in-oil, and oil-in-water-in-silicone emulsions.

In some embodiments, the disclosure provides a use of an mSCF polypeptide in the treatment of a human subject according to any of the methods disclosed herein.

### Illustrative Compositions

In one aspect, provided herein is a composition comprising an mSCF polypeptide. The mSCF polypeptide may be an mSCF polypeptide according to any of the various aspects and embodiments disclosed herein, including with respect to the various methods disclosed herein. In some embodiments, (i) the mSCF polypeptide is joined to a cell-penetrating peptide, or (ii) the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2. In some embodiments, the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3.

In some embodiments, the mSCF polypeptide comprises a sequence with at least 80% identity to 20 contiguous amino acids of SEQ ID NO: 1. In some embodiments, the mSCF polypeptide comprises (i) a sequence with between 80% to 99% identity to 20 contiguous amino acids of SEQ ID NO: 1, and (ii) one or more amino acid substitutions as compared to said 20 contiguous amino acids of SEQ ID NO: 1. In some embodiments, the one or more amino acid substitutions include one or more of D77H, K78E, K78D, K81I, V83E, D85Y, D85E, D85F, V87F, V87W, L88F, C89D, E92D, N93E, N93Q, N93D, A94P, P95D, P95K, P95V, N97I, N97K, 198K, 198D, 198N, 198E, K99D, K99E, K99I, K99W, K99T, K99Y, E100F, E100V, S101F, S101Y, S101D, S101E, P102K, P102F, P102Y, K103R, K103P, or R104Y, relative to SEQ ID NO: 1. In some embodiments, the mSCF polypeptide comprises a sequence with at least 80% identity to 20 contiguous amino acids of SEQ ID NO: 4. In some embodiments, the mSCF polypeptide comprises a sequence with at least 80% identity to SEQ ID NO: 4. In some embodiments, the mSCF polypeptide comprises the amino acid sequence of SEQ ID NO: 5. In some embodiments, the mSCF polypeptide comprises (i) a sequence with at least 80% sequence identity to any one of SEQ ID NOs: 15-36, 44, 58, or 59; or (ii) the sequence of any one of SEQ ID NOs: 15-36, 44, 58, or 59. In some embodiments, the mSCF polypeptide comprises (i) a sequence with at least 80% sequence identity to any one of SEQ ID NOs: 78-167; or (ii) the sequence of any one of SEQ ID NOs: 78-167. In some embodiments, the mSCF polypeptide is at least 20 amino acids in length, such as at least 30 amino acids in length. In some embodiments, the mSCF polypeptide is 20 amino acids to 75 amino acids in length, such as about 30 amino acids in length. In some embodiments, the mSCF polypeptide comprises at least 4 contiguous amino acids of SEQ ID NO: 29, such as 5 to 13 contiguous amino acids of SEQ ID NO: 29. In some embodiments, the mSCF polypeptide is selected from any one of SEQ ID NOs: 51-57. In some embodiments, the mSCF polypeptide consists of the amino acid sequence according to SEQ ID NO: 54, 55, or 57. In some embodiments, the mSCF polypeptide comprises one or more of a biotin group, a palmitoyl group, a norleucine, or an aspartic acid methyl ester. In some embodiments, the biotin group is joined to the mSCF polypeptide by a C11 linker.

In some embodiments, the mSCF polypeptide is joined to a cell-penetrating peptide, such as a cell-penetrating peptide comprising the sequence of SEQ ID NO: 6 or 7. In some embodiments, the mSCF polypeptide is joined to the cell-penetrating peptide by a linker sequence, such as a linker sequence comprising the sequence of SEQ ID NO: 8.

In some embodiments, the composition comprises the MSH polypeptide. In some embodiments, the MSH polypeptide is a β-MSH polypeptide or a γ-MSH polypeptide. In some embodiments, the composition comprises the Endothelin polypeptide. In some embodiments, the Endothelin polypeptide is an Endothelin-1 polypeptide or an Endothelin-3 polypeptide. In some embodiments, the composition comprises histamine. In some embodiments, the composition comprises two or more of the mSCF polypeptide, the MSH polypeptide, the Endothelin polypeptide, or histamine.

In some embodiments, the composition further comprises a delivery vehicle, wherein the delivery vehicle is effective to increase delivery of the mSCF polypeptide across the epidermis of a human subject. In some embodiments, the delivery vehicle comprises microneedles, liposomes, or a chemical penetration enhancer.

In one aspect, provided herein is a use of any of the compositions disclosed herein in the manufacture of a medicament. In some embodiments, the medicament is for use in treating a condition of a human subject. In some embodiments, the condition comprises melanocyte deficiency or dysfunction. In some embodiments, the condition comprises hair loss or hair whitening. In some embodiments, the medicament is formulated for topical delivery to the skin of a human subject.

In one aspect, provided herein is a polynucleotide encoding an mSCF polypeptide of the present disclosure. The mSCF polypeptide may be an mSCF polypeptide according to any of the various aspects and embodiments disclosed herein, including with respect to the various methods and compositions disclosed herein.

In another aspect, provided herein is a cell comprising a polynucleotide of the present disclosure. The mSCF polypeptide may be an mSCF polypeptide according to any of the various aspects and embodiments disclosed herein, including with respect to the various methods and compositions disclosed herein.

### Ranges

Throughout this disclosure, various aspects can be presented in range format. It should be understood that any description in range format is merely for convenience and brevity and not meant to be limiting. Accordingly, the description of a range should be considered to have specifically disclosed all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 2.1, 2.2, 2.5, 3, 4, 4.75, 4.8, 4.85, 4.95, 5, 5.5, 5.75, 5.9, 5.00, and 6. This applies to a range of any breadth.

### ILLUSTRATIVE EMBODIMENTS

The present disclosure provides the following illustrative embodiments.

Embodiment 1. A method of treating a condition of a human subject, the method comprising administering to the human subject a composition comprising one or more of a murine Stem Cell Factor (mSCF) polypeptide, an MSH polypeptide, or an Endothelin polypeptide.

Embodiment 2. The method of Embodiment 1, wherein the composition comprises the mSCF polypeptide.

Embodiment 3. The method of Embodiment 1 or 2, wherein the condition comprises melanocyte deficiency or dysfunction.

Embodiment 4. The method of any one of Embodiments 1-3, wherein the treating comprises one or more of: (i) inhibiting apoptosis of melanocytes or melanocyte stem cells (McSCs), (ii) increasing number or survival of melanocytes or McSCs, (iii) increasing a level of melanin synthesis, (iv) inhibiting hair whitening, (v) promoting increased hair pigmentation, (vi) inhibiting hair loss, or (vii) promoting increased hair growth; as compared to the absence of said treating.

Embodiment 5. The method of any one of Embodiments 1-4, wherein the administering comprises topical administration of the composition to the skin of the human subject.

Embodiment 6. The method of Embodiment 5, wherein the skin comprises the scalp or face of the human subject.

Embodiment 7. The method of any one of Embodiments 1-6, wherein said treating is effective to reduce a rate of hair whitening, or promote an increase in hair pigmentation in the subject.

Embodiment 8. The method of any one of Embodiments 2-7, wherein the mSCF polypeptide comprises a polypeptide of SEQ ID NO: 1, or a fragment or variant thereof.

Embodiment 9. The method of any one of Embodiments 2-8, wherein the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2.

Embodiment 10. The method of any one of Embodiments 2-9, wherein the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3.

Embodiment 11. The method of any one of Embodiments 2-10, wherein the mSCF polypeptide comprises a sequence with at least 80% identity to 20 contiguous amino acids of SEQ ID NO: 1.

Embodiment 12. The method of any one of Embodiments 2-10, wherein the mSCF polypeptide comprises (i) a sequence with between 80% to 99% identity to 20 contiguous amino acids of SEQ ID NO: 1, and (ii) one or more amino acid substitutions as compared to said 20 contiguous amino acids of SEQ ID NO: 1.

Embodiment 13. The method of Embodiment 12, wherein the one or more amino acid substitutions include one or more of D77H, K78E, K78D, K81I, V83E, D85Y, D85E, D85F, V87F, V87W, L88F, C89D, E92D, N93E, N93Q, N93D, A94P, P95D, P95K, P95V, N97I, N97K, 198K, 198D, 198N, 198E, K99D, K99E, K99I, K99W, K99T, K99Y, E100F, E100V, S101F, S101Y, S101D, S101E, P102K, P102F, P102Y, K103R, K103P, or R104Y, relative to SEQ ID NO: 1.

Embodiment 14. The method of any one of Embodiments 2-10, wherein the mSCF polypeptide comprises a sequence with at least 80% identity to 20 contiguous amino acids of SEQ ID NO: 4.

Embodiment 15. The method of any one of Embodiments 2-10, wherein the mSCF polypeptide comprises a sequence with at least 80% identity to SEQ ID NO: 4.

Embodiment 16. The method of any one of Embodiments 2-10, wherein the mSCF polypeptide comprises the amino acid sequence of SEQ ID NO: 5.

Embodiment 17. The method of any one of Embodiments 2-8, wherein the mSCF polypeptide comprises (i) a sequence with at least 80% sequence identity to any one of SEQ ID NOs: 15-36, 44, 58, or 59; or (ii) the sequence of any one of SEQ ID NOs: 15-36, 44, 58, or 59.

Embodiment 18. The method of any one of Embodiments 2-17, wherein the mSCF polypeptide is at least 20 amino acids in length; optionally wherein the mSCF polypeptide is at least 30 amino acids in length.

Embodiment 19. The method of Embodiment 18, wherein the mSCF polypeptide is 20 amino acids to 75 amino acids in length; optionally wherein the mSCF polypeptide is about 30 amino acids in length.

Embodiment 20. The method of any one of Embodiments 2-10, wherein the mSCF polypeptide comprises at least 4 contiguous amino acids of SEQ ID NO: 29; optionally wherein the mSCF polypeptide comprises 5 to 13 contiguous amino acids of SEQ ID NO: 29.

Embodiment 21. The method of Embodiment 20, wherein the mSCF polypeptide is selected from any one of SEQ ID NOs: 51-57.

Embodiment 22. The method of Embodiment 21, wherein the mSCF polypeptide consists of the amino acid sequence according to SEQ ID NO: 54, 55, or 57.

Embodiment 23. The method of any one of Embodiments 2-22, wherein the mSCF polypeptide comprises one or more of a biotin group, a palmitoyl group, a norleucine, or an aspartic acid methyl ester; optionally wherein the biotin group is joined to the mSCF polypeptide by a C11 linker.

Embodiment 24. The method of any one of Embodiments 2-23, wherein the mSCF polypeptide is joined to a cell-penetrating peptide; optionally wherein the cell-penetrating peptide comprises the sequence of SEQ ID NO: 6 or 7.

Embodiment 25. The method of Embodiment 24, wherein the mSCF polypeptide is joined to the cell-penetrating peptide by a linker sequence; optionally wherein the linker sequence comprises the sequence of SEQ ID NO: 8.

Embodiment 26. The method of any one of Embodiments 1-25, wherein the composition comprises the MSH polypeptide; optionally wherein the MSH polypeptide is a β-MSH polypeptide or a γ-MSH polypeptide.

Embodiment 27. The method of any one of Embodiments 1-26, wherein the composition comprises the Endothelin polypeptide; optionally wherein the Endothelin polypeptide is an Endothelin-1 polypeptide or an Endothelin-3 polypeptide.

Embodiment 28. The method of any one of Embodiments 1-27, wherein the composition further comprises a delivery vehicle, wherein the delivery vehicle is effective to increase delivery of the mSCF polypeptide across the epidermis of the human subject.

Embodiment 29. The method of Embodiment 28, wherein the delivery vehicle comprises microneedles, liposomes, or a chemical penetration enhancer.

Embodiment 30. Use of an mSCF polypeptide in the treatment of a human subject according to the method of any one of Embodiments 1-29.

Embodiment 31. A composition comprising an mSCF polypeptide, wherein (i) the mSCF polypeptide is joined to a cell-penetrating peptide, or (ii) the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2.

Embodiment 32. The composition of Embodiment 31, wherein the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3.

Embodiment 33. The composition of Embodiment 31 or 32, wherein the mSCF polypeptide comprises a sequence with at least 80% identity to 20 contiguous amino acids of SEQ ID NO: 1.

Embodiment 34. The composition of Embodiment 31 or 32, wherein the mSCF polypeptide comprises (i) a sequence with between 80% to 99% identity to 20 contiguous amino acids of SEQ ID NO: 1, and (ii) one or more amino acid substitutions as compared to said 20 contiguous amino acids of SEQ ID NO: 1.

Embodiment 35. The composition of Embodiment 34, wherein the one or more amino acid substitutions include one or more of D77H, K78E, K78D, K81I, V83E, D85Y, D85E, D85F, V87F, V87W, L88F, C89D, E92D, N93E, N93Q, N93D, A94P, P95D, P95K, P95V, N97I, N97K, 198K, 198D, 198N, 198E, K99D, K99E, K99I, K99W, K99T, K99Y, E100F, E100V, S101F, S101Y, S101D, S101E, P102K, P102F, P102Y, K103R, K103P, or R104Y, relative to SEQ ID NO: 1.

Embodiment 36. The composition of Embodiment 31 or 32, wherein the mSCF polypeptide comprises a sequence with at least 80% identity to 20 contiguous amino acids of SEQ ID NO: 4.

Embodiment 37. The composition of Embodiment 31 or 32, wherein the mSCF polypeptide comprises a sequence with at least 80% identity to SEQ ID NO: 4.

Embodiment 38. The composition of Embodiment 31 or 32, wherein the mSCF polypeptide comprises the amino acid sequence of SEQ ID NO: 5.

Embodiment 39. The composition of Embodiment 31 or 32, wherein the mSCF polypeptide comprises (i) a sequence with at least 80% sequence identity to any one of SEQ ID NOs: 15-36, 44, 58, or 59; or (ii) the sequence of any one of SEQ ID NOs: 15-36, 44, 58, or 59.

Embodiment 40. The composition of any one of Embodiments 31-39, wherein the mSCF polypeptide is at least 20 amino acids in length; optionally wherein the mSCF polypeptide is at least 30 amino acids in length.

Embodiment 41. The composition of Embodiment 40, wherein the mSCF polypeptide is 20 amino acids to 75 amino acids in length; optionally wherein the mSCF polypeptide is about 30 amino acids in length.

Embodiment 42. The composition of Embodiment 31 or 32, wherein the mSCF polypeptide comprises at least 4 contiguous amino acids of SEQ ID NO: 29; optionally wherein the mSCF polypeptide comprises 5 to 13 contiguous amino acids of SEQ ID NO: 29.

Embodiment 43. The composition of Embodiment 42, wherein the mSCF polypeptide is selected from any one of SEQ ID NOs: 51-57.

Embodiment 44. The composition of Embodiment 43, wherein the mSCF polypeptide consists of the amino acid sequence according to SEQ ID NO: 54, 55, or 57.

Embodiment 45. The composition of any one of Embodiments 31-44, wherein the mSCF polypeptide comprises one or more of a biotin group, a palmitoyl group, a norleucine, or an aspartic acid methyl ester; optionally wherein the biotin group is joined to the mSCF polypeptide by a C11 linker.

Embodiment 46. The composition of any one of Embodiments 31-45, wherein the mSCF polypeptide is joined to a cell-penetrating peptide; optionally wherein the cell-penetrating peptide comprises the sequence of SEQ ID NO: 6 or 7.

Embodiment 47. The composition of Embodiment 46, wherein the mSCF polypeptide is joined to the cell-penetrating peptide by a linker sequence; optionally wherein the linker sequence comprises the sequence of SEQ ID NO: 8.

Embodiment 48. The composition of any one of Embodiments 31-47, further comprising one or more of an MSH polypeptide, or an Endothelin polypeptide.

Embodiment 49. The composition of Embodiment 48, wherein the composition comprises the MSH polypeptide; optionally wherein the MSH polypeptide is a β-MSH polypeptide or a γ-MSH polypeptide.

Embodiment 50. The composition of Embodiment 48 or 49, wherein the composition comprises the Endothelin polypeptide; optionally wherein the Endothelin polypeptide is an Endothelin-1 polypeptide or an Endothelin-3 polypeptide.

Embodiment 51. The composition of any one of Embodiments 31-50, further comprising a delivery vehicle, wherein the delivery vehicle is effective to increase delivery of the mSCF polypeptide across the epidermis of a human subject.

Embodiment 52. The composition of Embodiment 51, wherein the delivery vehicle comprises microneedles, liposomes, or a chemical penetration enhancer.

Embodiment 53. Use of the composition of any one of Embodiments 31-52 in the manufacture of a medicament.

Embodiment 54. The use of Embodiment 53, wherein the medicament is for use in treating a condition of a human subject.

Embodiment 55. The use of Embodiment 54, wherein the condition comprises melanocyte deficiency or dysfunction.

Embodiment 56. The use of Embodiment 54, wherein the condition comprises hair loss or hair whitening.

Embodiment 57. The use of any one of Embodiments 53-56, wherein the medicament is formulated for topical delivery to the skin of a human subject.

Embodiment 58. A polynucleotide encoding the mSCF polypeptide according to any one of Embodiments 31-47.

Embodiment 59. A cell comprising the polynucleotide according to Embodiment 58.

### Materials and Methods for Examples 1-6

### Cell Lines

The Mnt-1 cell line, ordered from ATCC, was cultured in high-glucose DMEM supplemented with 10% FBS, 1% penicillin-streptomycin, and 1% glutamine, and maintained at 37° C. in a humidified atmosphere containing 5% CO₂. Cells were passaged at 70-80% confluence.

### In Vitro Induction Assay

To assay the ability of exemplary peptides, compounds or molecules to phosphorylate p42/RR, Mnt-1 cells were washed 3 times with pre-warmed PBS and incubated in starvation medium (growth medium without FBS) overnight. After starvation, cells were treated with candidate molecules for 5-10 minutes (unless otherwise specified) to detect p42/44 phosphorylation. For anti-apoptotic assays, cells were treated with either a wild-type mSCF polypeptide or the indicated mSCF polypeptide (identified by SEQ ID NO., also abbreviated as “SID” in the figures) for 2.5 hours, followed by 50 μg/ml ABT-199 (Ventoclax) for 1 hour prior to harvesting. Cells were harvested on ice using cold PBS, scraped, centrifuged to pellet the cells, and proteins were extracted using RIPA buffer in the presence of protease and phosphatase inhibitor.

### Western Blot

Cells were lysed in RIPA buffer containing protease and phosphatase inhibitors, followed by centrifugation at 12,000 g for 15 minutes at 4° C. Supernatants were collected, and protein concentrations were determined using the BCA assay. Equal amounts of protein were suspended in sample-loading buffer, heated to 95° C. for 5 minutes, and were resolved by SDS-PAGE and transferred to membranes. Membranes were blocked in 5% BSA filtered and supplemented with phosphatase inhibitors for 1 hour at room temperature, followed by overnight incubation at 4° C. with primary antibodies for phosphor-p42/44 MAPK, cleaved caspase-3, cleaved PARP, and beta-tubulin (“β-Tubulin”). After washing, membranes were incubated with HRP-conjugated secondary antibodies for 1 hour at room temperature. Protein bands were visualized using enhanced chemiluminescence (ECL) and detected using the Amersham ImageQuant 800 system. Illustrative results are shown in the immunoblot image analyses disclosed in the figures herein.

### Peptide Synthesis

Exemplary peptides of the present disclosure were synthesized by LifeTein, LLC. Peptides included various mSCF polypeptides disclosed herein, including 30-aa fragments with 15-aa overlaps, and fragments and variants thereof that were rationally designed to enhance activity of the Kit receptor in human cells. Mutations were introduced in mSCF polypeptides of SEQ ID NOs: 19 and 20, based on previously reported mSCF mutations. All peptides were purified to >95% purity by HPLC and verified by mass spectrometry. Water-soluble peptides were reconstituted in 0.1% BSA sterile water at 50 μg/ml and stored at −20° C. until use. Water-insoluble peptides were first dissolved in DMSO and then equal volume of water at concertation of 500 μg/ml.

### Example 1

The signaling potential of a murine SCF (mSCF) proteins in human cells was evaluated utilizing the human Mnt-1 cell line. Illustrative schematics of native murine SCF protein and its structural components at various stages is shown in FIG. 1, including the signal peptide (SP), extracellular domain (including the cleavage site, “Clv”), transmembrane domain (TMD), and cytosolic moiety of the top schematic. The middle schematic depicts an exemplary mature form of SCF (“Mature SCF”), which lacks the SP. The bottom schematic shows an exemplary cleaved, activated, and soluble fragment of the mature SCF protein (“Cleaved SCF”). The wild-type sequence of the soluble fragment of mSCF is presented in SEQ ID NO: 1.

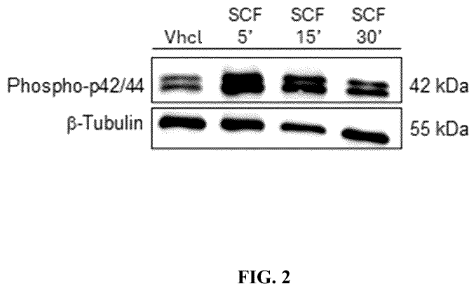

The phosphorylation of p42/44 MAPK by the mSCF protein over different time points was examined according to the “In vitro Induction Assay” and “Western Blot” methods described above. Mnt-1 cells were treated with 100 ng/ml of mSCF, and harvested after 5, 15, or 30 minutes. Phosphorylated p42/44 of treated cells was compared to that of cells treated with vehicle (0.1% BSA in PBS). Illustrative results are shown in FIG. 2. High activation of p42/44 MAPK was detected as early as 5 minutes post induction, which diminished after 30 minutes. The results demonstrated that mouse mSCF can activate the Kit receptor in human melanocytes, and that the Mnt-1 platform provides a robust model for detecting mSCF-mediated signal transduction.

### Example 2

Mnt-1 cells were used as a platform to screen for mSCF polypeptide variants and fragments capable of transducing the mSCF signal, as well as inhibiting cell death.

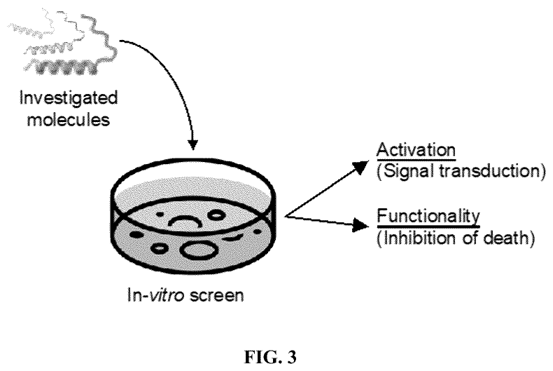

FIG. 3 shows an illustrative schematic representation of an Mnt-1-based platform that was used to screen mSCF polypeptides of interest using activity (p42/44 phosphorylation) and/or functionality (apoptotic markers) analysis.

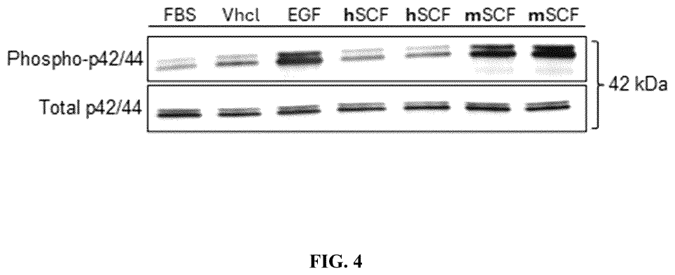

The effect of human SCF (hSCF) versus mSCF on human Mnt-1 cells was examined using the platform described above. In particular, each of hSCF and mSCF (100 ng/ml) were tested for their ability to induce phosphorylation of p42/44 in the human Mnt-1 cell line after 5-minutes induction. Negative controls were vehicle (Vhcl; 0.1% BSA in PBS), and 10% FBS, while the positive control for p42/44 phosphorylation was obtained with EGF (30 ng/mL). This study was performed in duplicates in order to account for potential variability in the findings. Illustrative results are shown in the immunoblot image in FIG. 4. The results show that mSCF was able to elicit a significantly stronger response than human SCF in human cells.

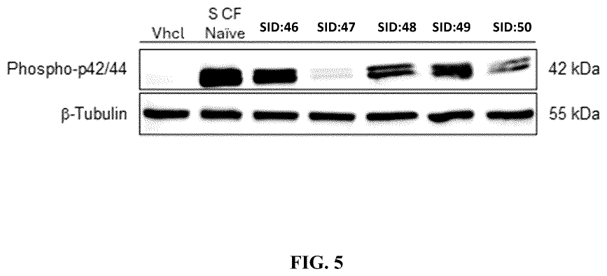

Several full-length soluble mSCF variants were also generated, and the activity thereof tested using the Mnt-1 cell platform. The full-length mSCF variants were tested for their ability to induce phosphorylation of p42/44 after 5 minutes of induction, and illustrative results are shown in FIG. 5. The mSCF variants were custom-made soluble mSCFs synthesized using a CHO expression system. “SCF Naïve” was a soluble mSCF provided by a local vendor. The mSCF polypeptide of SEQ ID NO: 46 was the CHO-produced wild-type/naïve protein. The mSCF polypeptide of SEQ ID NO: 47 contains 7 mutations reported to decrease specificity to progenitor cells in a hematopoietic system. The mSCF polypeptides of SEQ ID NOs: 48-50 are modifications SEQ ID NOs: 46 and 47 with a conjugated skin-penetrating peptide. Vehicle sample (Vhcl, 0.1% BSA in PBS) was used as a control. While mutations in 7 amino acid residues (as in the mSCF polypeptides of SEQ ID NOs: 47 and 50) exhibited lower activation levels, all variants were nonetheless able to induce p42/44 MAPK. The results show that mSCF polypeptides have a strong effect in human melanocytes.

### Example 3

This example describes the generation and evaluation of additional mSCF polypeptides.

30-amino acid (aa) fragments of mSCF were designed, giving rise to 10 peptides. Additionally, mSCF polypeptides of SEQ ID NOS: 19 and 20 were generated, which are mutated versions of the fragments according to SEQ ID NOs: 13 and 14, respectively. These mutations were reported to confer specificity toward hematopoietic stem cells and not activate mast cells, and were selected to test whether these mutations as reflected in SEQ ID NOs: 19 and 20 would confer similar specificity to melanocyte stem cells (McSCs) given their shared receptor, Kit. In addition to the 30-aa peptides, 50-aa peptides were developed based on mSCF sequences, deliberately incorporating regions implicated in SCF-Kit binding interference.

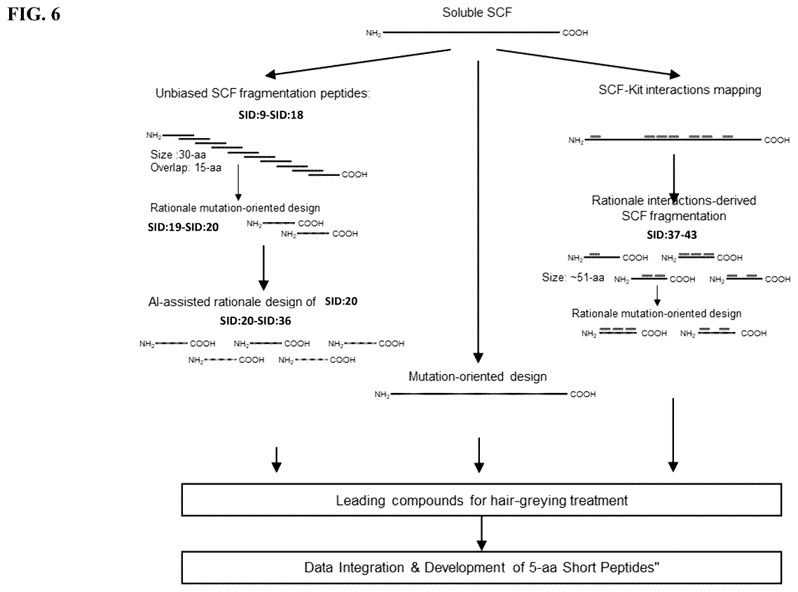

The overall development process is shown in the schematic of FIG. 6, which outlines three main strategies to develop candidates based on the native soluble mSCF: 1) initial fragmentation of soluble mSCF into 30-aa peptides with a 15-aa overlap, followed by targeted mutations and 2^{nd} generation, rationally-designed evolution; 2) mutations and modifications of full-length mSCF; and 3) design of 50-aa peptides focused on the SCF-Kit-interaction regions. The collective insights from a series of analyses were synthesized to design a 3rd generation of peptides, characterized by their remarkably short length and strong efficiency, as described in further detail herein.

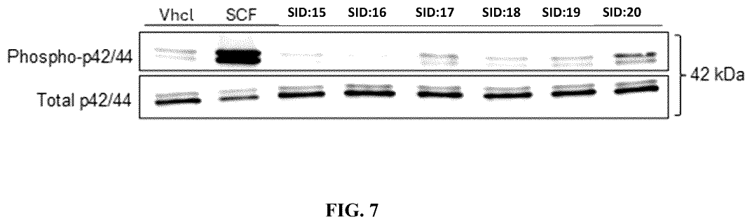

A series of 30-aa mSCF fragments with 15-aa overlaps between fragments were evaluated for activity. The 30-aa mSCF fragments (at 20 mg/ml) were tested for their ability to induce p42/44 phosphorylation after a 5-minute induction. Wild-type mSCF (at 100 ng/ml) served as a positive control, while vehicle (Vhcl, 0.1% BSA in PBS) was used as a negative control. Illustrative results for selected mSCF polypeptides are shown in FIG. 7. The mSCF polypeptide of SEQ ID NO: 20 (“SID: 20”), a variant of SEQ ID NO: 14 containing specific mutations, was identified as showing a mild level of activation. mSCF polypeptides of SEQ ID NOs: 9-14, which are not displayed in FIG. 7, did not exhibit any induction potential in these experiments.

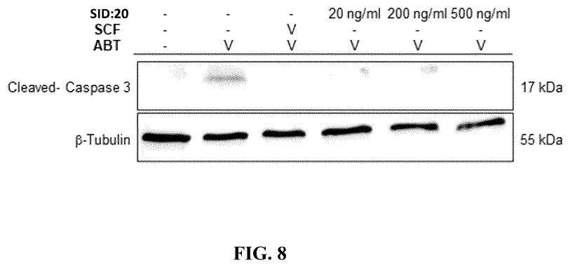

The mSCF polypeptide of SEQ ID NO: 20 was tested at various concentrations (20 ng/ml, 200 ng/ml, and 500 ng/ml) for its ability to reduce levels of the apoptotic marker cleaved caspase-3 (cCasp-3), a key mediator in the progression of apoptosis. Apoptosis was induced using the Bcl-2 inhibitor ABT-199 (at 50 μM). Cells were pre-treated with the mSCF of SEQ ID NO: 20, wild-type mSCF (positive control), or vehicle (negative control) for 2.5 hours, followed by a 1-hour exposure to ABT-199 (ABT) before harvesting for analysis. Illustrative results are shown in the immunoblot of FIG. 8 (“V” indicates addition of the indicated compound). Results show that the short mSCF polypeptide of SEQ ID NO: 20, even at low concentrations, countered ABT activity and significantly decreased levels of cleaved caspase-3 (cCasp-3), with results similar to those seen with native soluble mSCF. The ability to reduce cCasp-3 highlights the anti-apoptotic effect of the short mSCF polypeptide, in addition to its pro-survival role.

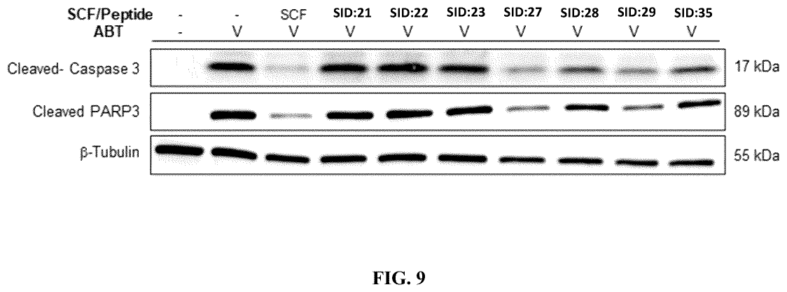

Based on the above findings, an illustrative set of second-generation peptides based on the mSCF polypeptide of SEQ ID NO: 20 was developed. The resulting mSCF polypeptides were tested for their ability to reduce levels of both cCasp-3 and cleaved PARP (cPARP), a substrate of cCasp-3 whose cleavage indicates active apoptosis. Apoptosis was induced using the Bcl-2 inhibitor ABT-199 (at 50 μM). Cells were pre-treated with an indicated mSCF polypeptide, wild-type soluble mSCF (positive control), or vehicle (negative control) for 2.5 hours in a similar manner as above, followed by a 1-hour exposure to ABT-199 before harvesting for analysis. Illustrative results are shown in FIG. 9 (with selected mSCF polypeptides indicated by SEQ ID NO, abbreviated as “SID”; wild-type control as “SCF”). Among the second-generation peptides, the mSCF polypeptides according to SEQ ID NOs: 27 and 29 were particularly effective in decreasing levels of both cCasp-3 and cPARP.

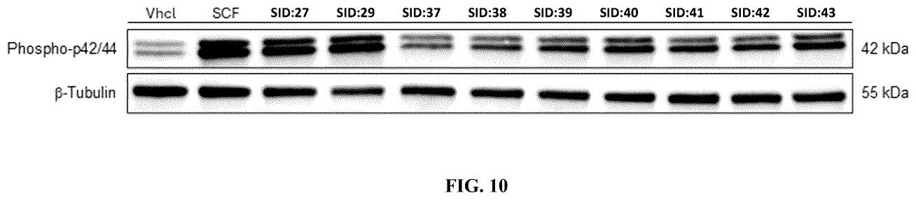

Selected mSCF polypeptide candidates were further tested at a higher concentration (1 μg/ml), including the 50-aa mSCF variants developed from the structural-guided SCF-Kit interaction analysis shown in FIG. 6. These peptide variants were designed to mimic regions involved in SCF-Kit binding, enhancing their potential to replicate the native mSCF's biological effects. The candidates were tested for their ability to induce p42/44 phosphorylation after a 5-minute induction (at (1 μg/ml) in a similar manner as set forth above. Wild-type mSCF (at (100 ng/ml) served as a positive control, while the vehicle (Vhcl, 0.1% BSA in PBS) was used as a negative control. Illustrative results are shown in the immunoblot of FIG. 10 (with selected mSCF polypeptides identified by “SID”, as above). In this set of experiments, it was demonstrated that both the second-generation mSCF polypeptides according to SEQ ID NOs: 27 and 29, and the 50-aa mSCF variants induced significant reductions in cCasp-3 and cPARP levels, reinforcing their anti-apoptotic effects. Using a higher concentration yielded results that closely resembled the effects of native mSCF, confirming the ability of these peptides to engage the Kit receptor effectively and block apoptosis through the same downstream pathways. The results are also consistent with SCF binding to the Kit receptor to activate survival pathways such as the MAPK and PI3K-Akt signaling cascades, which inhibit apoptotic processes by downregulating caspase activation and maintaining cellular integrity. Furthermore, the 50-aa variants, designed specifically to include regions of SCF-Kit interaction, demonstrated a technical effect on Mnt-1 cells comparable to that of full-length soluble mSCF. These results validate the design approach and highlight the potential for mSCF polypeptides described herein to serve as active agents for preserving melanocyte stem cell function and preventing apoptosis, particularly in the context of, e.g., hair pigmentation and skin regeneration.

### Example 4

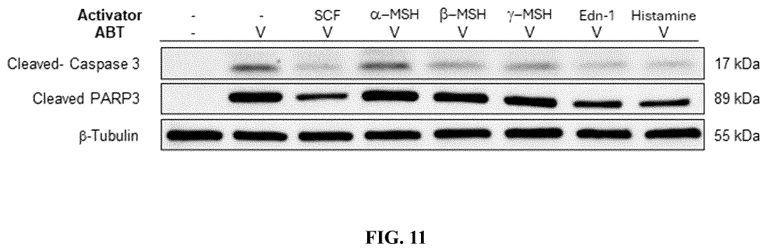

Additional non-SCF candidate molecules, including beta-MSH (β-MSH), gamma-MSH (γ-MSH), Endothelin 1 (Edn-1), and Histamine, were investigated in a similar manner as the preceding examples for their potential effect on human Mnt-1 melanocytes, illustrative results for which are shown in the immunoblot of FIG. 11. β-MSH and γ-MSH activate pathways like MAPK and cAMP/PKA, promoting melanocyte survival. As shown in FIG. 11, both molecules significantly reduced cCasp-3 and cPARP levels in Mnt-1 cells, indicating strong anti-apoptotic effects.

Endothelin 1 (Edn-1), acting through EDNRB, is linked to melanocyte survival via the PI3K-Akt pathway. Edn-1 demonstrated a robust reduction in apoptotic markers, aligning with its role in promoting melanocyte survival.

Histamine, although primarily associated with immune responses, also showed a noticeable decrease in cCasp-3 and cPARP levels, suggesting the signaling may intersect with melanocyte survival pathways.

Overall, the results in FIG. 11 show that β-MSH, γ-MSH, Edn-1, and Histamine possess significant SCF-like anti-apoptotic effects.

### Example 5

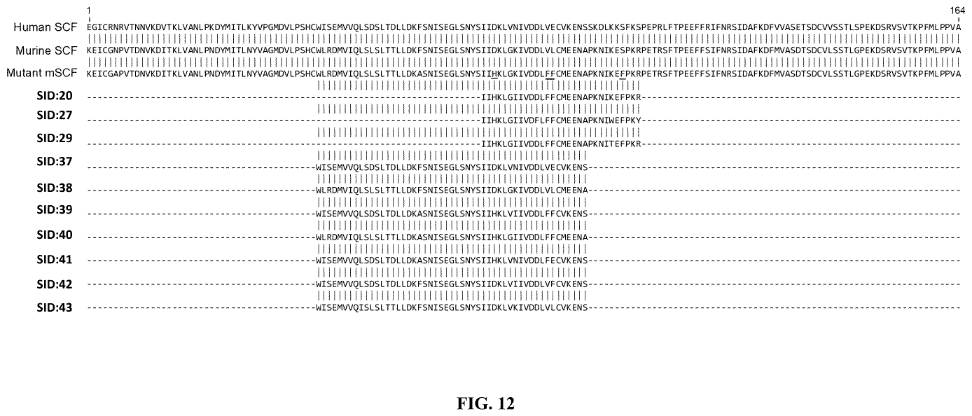

A sequence alignment was performed between human SCF and mSCF, along with the mutated forms of mSCF designed for the studies herein. An illustrative alignment is shown in FIG. 12. For the full-length “Human SCF” and “Murine SCF”, the sequence represents the corresponding 165-aa post-cleavage soluble SCF. The human sequence is shown as EGICRNRVTNNVKDVTKLVANLPKDYMITLKYVPGMDVLPSHCWISEMVVQLSDSLTD LLDKFSNISEGLSNYSIIDKLVNIVDDLVECVKENSSKDLKKSFKSPEPRLFTPEEFFRIFN RSIDAFKDFVVASETSDCVVSSTLSPEKDSRVSVTKPFMLPPVA (SEQ ID NO: 74). The murine SCF corresponds to SEQ ID NO: 1. The “Mutant mSCF” refers to a full-length soluble mSCF with several of the aforementioned 7 mutations of SEQ ID NO: 47, represented by the sequence KEICGAPVTDNVKDITKLVANLPNDYMITLNYVAGMDVLPSHCWLRDMVIQLSLSLTT LLDKASNISEGLSNYSIIHKLGKIVDDLFFCMEENAPKNIKEFPKRPETRSFTPEEFFSIFNR SIDAFKDFMVASDTSDCVLSSTLGPEKDSRVSVTKPFMLPPVA (SEQ ID NO: 75). Mutations in SEQ ID NO: 75 relative to SEQ ID NO: 1 that are also reflected in SEQ ID NO: 20 are underlined in FIG. 12.

By aligning the SCF sequences, differences between species were identified, and the conservation of specific functional domains was observed, particularly in the regions involved in Kit receptor binding. The mSCF polypeptide according to SEQ ID NO: 20, along with its derivatives according to SEQ ID NOs: 27 and 29, was aligned with the 50-aa polypeptides according to SEQ ID NOs: 37-43, revealing how these variants preserve or alter certain binding motifs.

Without wishing to be bound by theory, the alignment suggested that mutations reflected in the mSCF polypeptides according to SEQ ID NO: 19 and/or 20 may enhance their specificity towards McSCs by mimicking regions of SCF-Kit interaction. It was hypothesized that the mSCF polypeptide according to SEQ ID NO: 20 could naturally bind to Kit receptor in a manner that mimics the full-length mSCF protein.

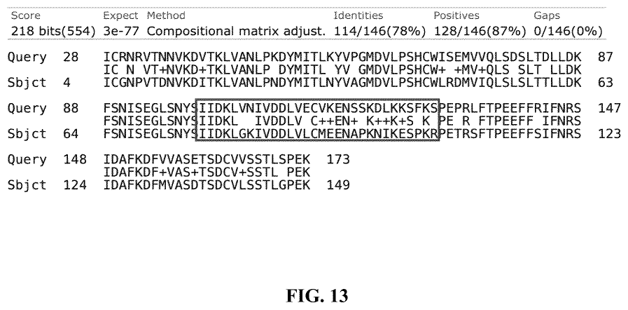

Notably, the main region in which hSCF and mSCF differ is specifically the region of SEQ ID NO: 20 and its derivatives (e.g., SEQ ID NOs: 27 and 29), as shown in FIG. 13. Specifically, FIG. 13 shows the alignment of portions of human (upper) and murine (lower) SCF sequences from the N-terminus, corresponding to the majority of soluble SCF moiety. The aligned portion of the human sequence is ICRNRVTNNVKDVTKLVANLPKDYMITLKYVPGMDVLPSHCWISEMVVQLSDSLTDLL DKFSNISEGLSNYSIIDKLVNIVDDLVECVKENSSKDLKKSFKSPEPRLFTPEEFFRIFNRSI DAFKDFVVASETSDCVVSSTLSPEK (SEQ ID NO: 76), and the aligned portion of the murine sequence is ICGNPVTDNVKDITKLVANLPNDYMITLNYVAGMDVLPSHCWLRDMVIQLSLSLTTLL DKFSNISEGLSNYSIIDKLGKIVDDLVLCMEENAPKNIKESPKRPETRSFTPEEFFSIFNRSI DAFKDFMVASDTSDCVLSSTLGPEK (SEQ ID NO: 77). The sequences appearing between SEQ ID NOs: 76 and 77 in the alignment represent those portions of SEQ ID NO: 76 that match corresponding positions of SEQ ID NO: 77. The region corresponding to SEQ ID NO: 14 and its mutated variants (e.g., SEQ ID NOs: 20-36) is highlighted by a box in FIG. 13, illustrating the significant sequence divergence between human and murine SCF in this region. It was hypothesized that the differences in this boxed region may be the underlying cause for the stronger signal elicited by mSCF in human melanocytes (as compared to human SCF).

### Example 6

3D remodeling of the solved human Kit structure in a complex with SCF was performed. After computationally removing SCF from the complex, the specific regions of the Kit receptor that are important for binding were examined.

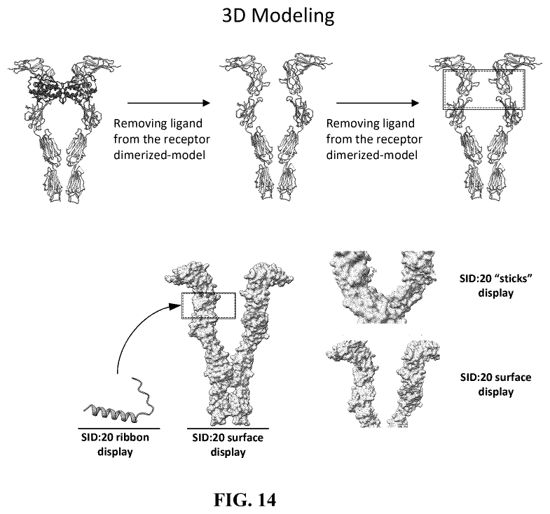

FIG. 14 shows structural modelling illustrating the structural features and binding interactions of SCF, focusing on the regions involved in c-Kit receptor interaction. The top row shows a comparative structural modeling of human SCF-c-Kit interactions, highlighting key binding regions. The left panel of the top row shows SCF bound to Kit. The middle panel of the top row presents the Kit structure without the SCF, as a basis for molecular docking simulation, while the right panel of the top row highlights the specific binding interface, boxed in a block, in which the docking simulation was tested. The bottom row shows a detailed view of the mSCF polypeptide according to SEQ ID NO: 20 binding on c-Kit. The leftmost panel of the bottom row displays a 3D structure of the mSCF polypeptide according to SEQ ID NO: 20. The middle panel of the bottom row provides a surface display of c-Kit with a zoomed-in view of the interaction site, boxed for clarity. The right panels of the bottom row further illustrate the binding conformation of the mSCF polypeptide according to SEQ ID NO: 20 with the c-Kit binding pocket, with the mSCF polypeptide shown in blue, emphasizing its position and orientation within the receptor's active site.

The in-silico analysis confirmed that the mSCF polypeptide according to SEQ ID NO: 20 was able to naturally bind within an important region of the Kit receptor. This binding pattern closely resembled the native interaction between SCF and Kit, supporting the notion that the mSCF polypeptide could act as an SCF-mimetic. This finding also provides a basis for using SID: 20 (and its derivatives or alternative peptides) as candidate compounds in modulating melanocyte lineage cell activation through Kit receptor engagement, which is central to, e.g., pigmentation processes and hair follicle function.

### Example 7

mSCF polypeptides with further reduced lengths were designed to test whether penetration ability and/or efficiency could be improved thereby.

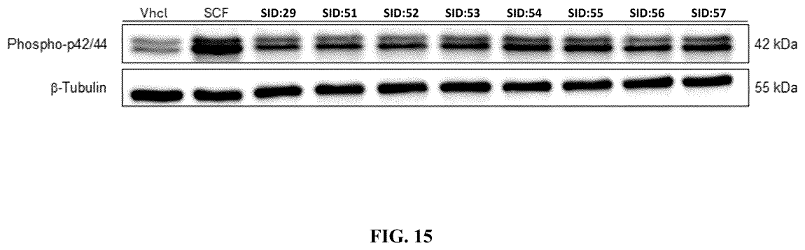

Building upon the studies described above, including insights from the best-performing 30-aa candidates, structural-guided design, and sequence alignment of top candidates, shorter peptides ranging from 5 to 13 amino acids were designed and analyzed in a similar manner as the “peptide synthesis”, “In vitro Induction Assay”, and “Western Blot” methods described above. These “3^{rd} generation” short peptides, based on the evolution of the 30-aa mSCF polypeptide according to SEQ ID NO: 29, were tested for their ability to induce p42/44 phosphorylation after a 5-minute induction (1 μg/ml). Wild-type mSCF (100 ng/ml) served as a positive control, while the vehicle (Vhcl, 0.1% BSA in PBS) was used as a negative control. Illustrative results are shown in the immunoblot image of FIG. 15. These shorter peptide designs demonstrated notable phosphorylation of p42/44 MAPK.

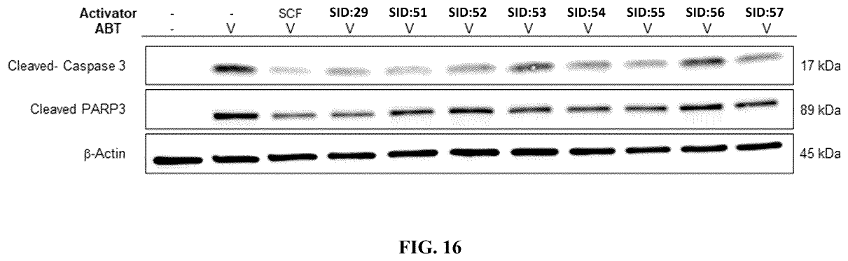

These short peptides were also tested for their ability to reduce levels of cCasp-3 and cPARP, mimicking a SCF-like effect. Apoptosis was induced using the Bcl-2 inhibitor ABT-199 (50 μM). Cells were pre-treated with an activator, mSCF, or Vehicle for 2.5 hours as indicated above, followed by a 1-hour exposure to ABT-199 before harvesting for analysis. Illustrative results are shown in FIG. 16.

While activity was observed to some degree across all tested candidates, the 5-aa peptides according to SEQ ID NOs: 54, 55, and 57 exhibited exceptional performance. These peptides not only induced robust signaling (as shown in FIG. 15), but also displayed SCF-like anti-apoptotic effects, as evidenced by the immunoblot analysis of cCASP-3 and cPARP following apoptotic stimuli (as shown in FIG. 16).

These results further validate the design approach and highlight the potential for mSCF polypeptides described herein to serve as active agents for preserving melanocyte stem cell function and preventing apoptosis, particularly in the context of, e.g., hair pigmentation and skin regeneration.

### Example 8

Additional experiments using mSCF polypeptides described herein are conducted to show activity in human skin and hair follicle explants. These models more closely emulate the in vivo environment of human skin, providing a more accurate assessment of the mSCF polypeptides' effects on melanocyte lineage activation and survival. mSCF polypeptides, such as illustrative polypeptides described in the example above, are applied to the model. Activity is shown by a positive impact on the survival of and/or activation in the melanocytic linage within human hair follicles in the model. Activity in the model will serve to further validate the therapeutic potential of the mSCF polypeptides in enhancing pigmentation and promoting melanocyte function in human tissue.

### Example 9

A dataset of 90 peptide sequences, each sharing at least 80% homology with the reference sequence IIDKLGKIVDDLVLCMEENAPKNIKESPKR (SEQ ID NO: 14, a 30-aa sequence from the Murine SCF's interacting site), was computationally generated and subjected to virtual screening for binding affinity to the human stem cell factor (SCF) receptor (c-Kit). Molecular docking simulations were performed using AutoDock CrankPep, a tool optimized for flexible peptide docking, to evaluate the interaction of each peptide-receptor complex. The analysis provided key binding metrics, including cluster size (reflecting docking stability) and binding affinity scores, enabling the identification of candidate peptides with enhanced receptor interaction profiles. Potential candidates were selected for further experimental validation based on their predicted stability and binding strength within the SCF-Kit interaction site.

The mSCF polypeptides identified are provided in Table 2 below.

TABLE 2
Illustrative mSCF polypeptides

| | Affinity | Cluster | SEQ ID |
|---|---|---|---|
| Sequence | (kcal/mol) | Size | NO: |
| IIDKLYYIVDHLVLCMEENAPKNIKERPKR | −9.8 | 72 | 78 |
| IITKLGKIVDDLVLCMEENWDKNDKESFKR | −7.7 | 48 | 79 |
| IIDKLGKIVQDLVSCMEEWAMINIKESPKR | −7.6 | 7 | 80 |
| IIDKLGFIVDGLVLCMEENAPKNIKESPGR | −7.5 | 25 | 81 |
| IIDKLGKIHDDLVLCMEENAPTTIKESPKR | −7.2 | 31 | 82 |
| IIHKLGIIVDDLFFCMEENAPKNIKEFPKR | −7.2 | 23 | 83 |
| IIDKLGKKVDDLYLYMEELAPKNITEGPKR | −6.8 | 8 | 84 |
| IIYSLGKIVDDLVLCMEEPAPDNIKESWKR | −6.8 | 6 | 85 |
| IIEKLGSGVDDLVLCMEENAVKNMKSSPKR | −6.7 | 23 | 86 |
| EIDKKGKIDDDLVLFSEENAPKNIKESPKR | −6.6 | 18 | 87 |
| IIWTLGKIVDLLHLCMEENAPKNISESPKR | −6.5 | 29 | 88 |
| IIGKCGKISRDLVLCMEENAPKNEKESKKR | −6.2 | 12 | 89 |
| IIDKLGKICDDLVLWMEENAPANIKESPKR | −5.9 | 19 | 90 |
| IIDKLLKIVDDLVDCMELWAPKNIKESPKR | −5.8 | 24 | 91 |
| IIDKWGKIVDDLPLCMEYNAIKQIKESPKR | −5.8 | 23 | 92 |
| IIDKLGKIVMDLVNCMEENAMKNIKESPTR | −5.7 | 14 | 93 |
| IIDKLGSIVDDLVVCMEENAPKNIKESPKL | −5.6 | 29 | 94 |
| IIDKLGVIVDDLVLCMEENAPKSIKESPKR | −5.6 | 25 | 95 |
| IIDKCGKCVDDVVLCMESNAPKNIKRSDKR | −5.4 | 23 | 96 |
| IIDMLGKIVDDLVCCMEENMPKNIKESPKR | −5.4 | 15 | 97 |
| IIWKLGKIVDDLVLCMEYNAPKNIKESPKR | −5.4 | 27 | 98 |
| MIDKLGKIVDDLVLCMEENAPKVIVESPKS | −5.3 | 13 | 99 |
| ACDKLGKKADDLMLCMEENAPKNIKNSPKR | −5.2 | 17 | 100 |
| IIDALPKIVDDLVLCMEEYAPKNIKESPKR | −5.2 | 11 | 101 |
| IIWKLGKIVDDLVLCMEENAPKKTKRSPKR | −5.2 | 12 | 102 |
| IIDKLGKIWDDLVLCMEMYAPKNIKESPKR | −5.1 | 8 | 103 |
| FSDKLMKIVHDLVLCMEEPAPKNIKESPKR | −4.9 | 20 | 104 |
| IVVKLGKIVHDLPQCMEENAPKNIKSSPKR | −4.8 | 27 | 105 |
| KEDKLGKIVDDLMLCMEENAPKNIFESEPR | −4.8 | 11 | 106 |
| IIHKLGIIVDFLFFCMEENAPKNIWEFPKY | −4.8 | 35 | 107 |
| ICDYYHKIVDDLVLCTEENAPKNIKELPKR | −4.7 | 37 | 108 |
| IKDALGKEVDDLGLYMEENAPKNIKESPMR | −4.7 | 14 | 109 |
| IIDKLGQIVDDLVCNMEENAPKDIKESPKM | −4.5 | 27 | 110 |
| IIDELGKIVDDHVLCMEENAPQNIKESPKL | −4.5 | 24 | 111 |
| IGDKLGDIVDDLVLCMEENAPENIKESPKR | −4.4 | 21 | 112 |
| IIDKLGKITDDLVLCMEEAAPKNIKEQPKR | −4.4 | 13 | 113 |
| IIDKLNKIVDLLVLCMEINAPKNIKWSPKR | −4.3 | 18 | 114 |
| IQDKLVKIVDDLVLCMESNAPKNIKESPKR | −4.3 | 8 | 115 |
| IIDKMGKIREDLVLCMEENMPKNIKESPKR | −4.2 | 24 | 116 |
| IIDNLGKIVDLLVLCMEENAPKNIKESPKR | −4.2 | 40 | 117 |
| IIDKVGKIKDDLVLCMEENAPKNFKESPKR | −4.1 | 14 | 118 |
| IIDSLGNGVDDLGLCMEENAPKNIKESPKR | −4.1 | 10 | 119 |
| IIDKLLKIVDDLVLCMEEIAPKNIKEFPKR | −4 | 10 | 120 |
| IIDKLQKIVDDLVLCMELNWPKNIKEKPKR | −3.9 | 7 | 121 |
| IIDYLKKAVDDLVLCMEENAPKNIKESPFR | −3.9 | 17 | 122 |
| IINKLGKIMDDQVLCMEERAPKLIKESPCR | −3.9 | 19 | 123 |
| IIDCLGKIVDDLVLEMEENAPKNSKESPLI | −3.8 | 29 | 124 |
| ICDKLGKIVDDLVLCMEENAPKNIKESPKR | −3.7 | 26 | 125 |
| IIDFLCKIWDDMVLCMEENAPKNIKESYKR | −3.7 | 21 | 126 |
| IIDKLGKIVKDLVLCMEEDAPKNIKESPKR | −3.7 | 10 | 127 |
| KIDKDGKIVDDLVLCMEENAPKFIWESPKR | −3.7 | 25 | 128 |
| PIDKLGKINDDLVLCMEENAPKNTKESPKR | −3.7 | 20 | 129 |
| IKLKLGKITDDLVCCMEEMAPKNIKESPKR | −3.6 | 21 | 130 |
| FIDKLVKIEDDLVLCMEENAPKNIKESPKA | −3.4 | 25 | 131 |
| IIDGLGKIKDDLALCMEYNAPKNIIESPKR | −3.4 | 19 | 132 |
| IIDKEGKKVDDLVLCMEENAPKNIKESPNK | −3.4 | 17 | 133 |
| IIDKLGKIVDDLVLCMEERAPKNIKESPKR | −3.4 | 23 | 134 |
| MIDKLMKTVDDLVLCMEANAPKDIKESPKR | −3.4 | 6 | 135 |
| IIDKLGKIVDDLVVCMEENAPKSIKESPKR | −3.3 | 24 | 136 |
| IIDKLGKIVSDLVLCMEENAPKNIGESPKR | −3.2 | 5 | 137 |
| IIDKLGKIVTDVVLCMEEFAPKNIKHSPHM | −3.2 | 21 | 138 |
| IIDKPGNIVDDAVVCMEENAPKNIKESVKR | −3.2 | 16 | 139 |
| IIDKLRKIVIDLVLCYPGNAVKNIKESPKR | −3.1 | 27 | 140 |
| IIDKQGKIVLCLVLGMEEWAPKNTKESPKR | −3.1 | 14 | 141 |
| IIDKWGKIVDDAVHCMEENAPWNIIISPKR | −3.1 | 26 | 142 |
| IIDQLIKIVDDLVLNMEENAPKNTKESPNR | −3.1 | 26 | 143 |
| IQDSLGKIVPDLVLCWEENAPKNIKESPKR | −3 | 14 | 144 |
| IIDKLGKIVFLAVLCMEENHPKNIKESPKR | −2.9 | 24 | 145 |
| LRYALGKIVDDLVLCMEENAPQNIRESPKR | −2.8 | 25 | 146 |
| IIDELGKIVDDLVQCMEENAPHVIKESPKR | −2.7 | 14 | 147 |
| IIDKLDTIVDNLVLCMEENAPKNITESPKM | −2.7 | 14 | 148 |
| IIDLLGRIVDDLDSCMEENPPKNIKESPKR | −2.7 | 29 | 149 |
| SIDKKGFIVDDLVHCMEENPPKNIHESPKR | −2.7 | 12 | 150 |
| IIHKQGKIKDGLVLCMCESAPKNIKESPKR | −2.6 | 5 | 151 |
| RIDKLRKIVHDLVLRMFEGAPKNIKESPKR | −2.5 | 16 | 152 |
| IIHKLGIIVDDLFFCMEENAPKNITEFPKR | −2.5 | 22 | 153 |
| IIDKHGFIVDDPVLCCEENAPKNIKESTKR | −2.3 | 10 | 154 |
| IIDKLAKIVDDLVFCMEEQAPKNSMESPFR | −2.3 | 17 | 155 |
| IIDKPGKAVDDLVLCMGENAPKNIKESPKR | −2.3 | 16 | 156 |
| IIDRNGKIVDDLVLCMEENAPRNIKESPKR | −2.3 | 22 | 157 |
| IIDKIGKIVDDLCLCMEENAPKNIKESPKR | −2.2 | 10 | 158 |
| IIDKLGCIVDDLGLCMEENAPKAIKESPKD | −2.2 | 20 | 159 |
| IIKKLGTIVDDLVLCMEENAPVNNKEAPMR | −1.8 | 11 | 160 |
| PIAKLGTIVDDNVGCMEENAPKFIKESPKR | −1.8 | 15 | 161 |
| VIDKLGKIVDPLVLCMLENAPKLIKVSPKR | −1.8 | 8 | 162 |
| IIDKLGKIVADPVLCMEAYAPKNIKVSPKR | −1.7 | 14 | 163 |
| IHDKLGKILDDLVHCMEENMLKNIKESPKN | −1.4 | 16 | 164 |
| IIDKPGKIVDDLVLCMEENQPENIKESPMR | −1.4 | 14 | 165 |
| FIDKLGKIVDDLVLCMEPNWPKEIKESPKR | −1.1 | 17 | 166 |
| IIDKLGKIVKQLVLCMWENAPKNTKESPKR | −0.2 | 6 | 167 |

### Example 10

To identify chemical modifications capable of enhancing the properties (e.g., stability, activity, and penetration) of mSCF polypeptides, various chemical modifications to selected mSCF polypeptides were made. For illustrative purposes, modifications were made to the mSCF polypeptides according to SEQ ID NOs: 53-55, 58, and 59. These modifications were evaluated in our in-vitro model, where they demonstrated a substantial impact on peptide function.

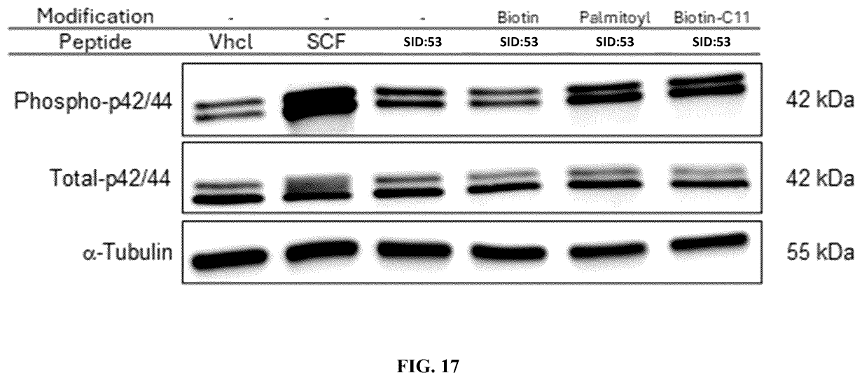

Various modifications of the mSCF polypeptide according to SEQ ID NO: 53 were tested for their ability to induce p42/44 phosphorylation. Wild-type mSCF (“SCF”) served as a positive control, while the vehicle (“Vhcl”) was used as a negative control. Illustrative results are shown in the immunoblot of FIG. 17, with the selected mSCF polypeptides identified by “SID” (as above), and modification thereof identified above the modified mSCF polypeptide, where applicable. Tested modifications included Biotinylation, Palmitoylation, and Biotin linked to a C11 spacer (“Biotin-C11”). Varied effects on peptide activity were observed. The results confirmed that these modifications modulate the functional properties of mSCF peptides and can be tailored to optimize their bioactivity and delivery efficiency.

### Example 11

A Helsinki-approved human hair follicle explants model, obtained from hair transplantation procedures, was utilized to assess activity of mSCF polypeptides in a physiologically relevant model. This ex vivo model closely mimics the human skin microenvironment, allowing for a more accurate evaluation of biological effects. The model was used to test mSCF polypeptides according to SEQ ID NOs: 20, 27, 29, 46, 53-55, and 60-73 (with the modifications as indicated in Table 1 for SEQ ID NOs: 60-73). An explant was treated with an mSCF polypeptide in vehicle, a combination of mSCF polypeptides in vehicle, or vehicle alone for 16 hours. The vehicle solution for these experiments was prepared by adding 2 μl of 0.1% BSA and 6 μl 1:1 PBS: DMSO to 1 mL of culture medium. After mSCF/vehicle treatment, explants were treated with 100 μM of ABT-199 for 2 hours. After treatment, samples were fixed in 4% paraformaldehyde (PFA) for 40 minutes, followed by three phosphate-buffered saline (PBS) washes. Samples then underwent a sucrose gradient (10% to 30%) before cryopreservation in optimal cutting temperature (OCT) medium for subsequent cryosectioning and immunofluorescence analysis. Cryopreserved samples were sectioned and analyzed via immunofluorescence using DAPI (to stain nuclei), MITF (to label the melanocytic lineage), and Cleaved Caspase-3 (cCP3) as a marker of apoptosis.

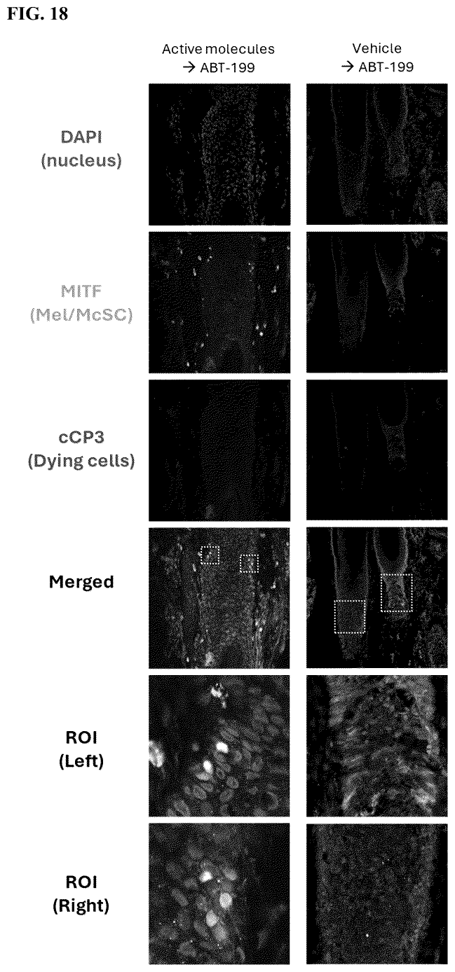

Using individual compounds or specific mixtures, preliminary results indicated that mSCF polypeptides effectively block destructive apoptotic signals, such as those induced by Bcl-2 inhibition (ABT-199), and promote the survival of melanocyte stem cells. Illustrative imaging results for an explant treated with (1) a combination of mSCF polypeptides followed by ABT-199, compared to (2) an explant treated with vehicle followed by ABT-199 are shown in FIG. 18. The combination of mSCF polypeptides included mSCF polypeptides according to SEQ ID NOs: 20, 27, 29, an 46. This ex vivo approach provides key insights into the mechanisms of melanocyte protection and survival, further supporting the technical effects observed in the in vitro models.

Unless defined otherwise, all technical and scientific terms used herein have the same meaning as is commonly understood by one of skill in the art to which this disclosure belongs.

Any and all references and citations to other documents, such as patents, patent applications, patent publications, journals, books, and papers, that have been made throughout this disclosure are hereby incorporated herein in their entirety for all purposes. Although embodiments of the invention have been described with reference to the above examples, it will be understood that modifications and variations are encompassed within the spirit and scope of the invention.

1. A method of treating a condition of a human subject, the method comprising administering to the human subject a composition comprising one or more of a murine Stem Cell Factor (mSCF) polypeptide; wherein:
(a) the mSCF polypeptide: (i) is 5-60 amino acids in length; and (ii) comprises the amino acid sequence of any one of SEQ ID NOs: 15-36 or 51-73; and
(b) the treating comprises one or more of: (i) inhibiting apoptosis of melanocytes or melanocyte stem cells (McSCs), (ii) increasing number or survival of melanocytes or McSCs, (iii) increasing a level of melanin synthesis, (iv) inhibiting hair whitening, (v) promoting increased hair pigmentation, (vi) inhibiting hair loss, or (vii) promoting increased hair growth; as compared to the absence of said treating.

2. The method of claim 1, wherein the the mSCF polypeptide is 5-30 amino acids in length and comprises the amino acid sequence of any one of SEQ ID NOs: 15-36 or 51-73.

3. The method of claim 1, wherein the mSCF is 5-20 amino acids in length and comprises the amino acid sequence of any one of SEQ ID NOs: 51-57, 60-65, or 68-73.

4. The method of claim 1, wherein the treating comprises: (i) inhibiting apoptosis of melanocytes or melanocyte stem cells (McSCs), or (ii) increasing number or survival of melanocytes or McSCs; as compared to the absence of said treating.

5. The method of claim 1, wherein the administering comprises topical administration of the composition to the skin of the human subject; optionally wherein the skin comprises the scalp or face of the human subject.

6. (canceled)

7. The method of claim 1, wherein said treating is effective to reduce a rate of hair whitening, or promote an increase in hair pigmentation in the subject.

8. The method of claim 1, wherein the treating comprises increasing a level of melanin synthesis, as compared to the absence of said treating.

9. The method of claim 1, wherein the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2.

10. The method of claim 1, wherein the mSCF polypeptide comprises a C-terminal truncation of at least 10 amino acids as compared to SEQ ID NO: 1, such that the mSCF polypeptide does not comprise 50 contiguous amino acids with more than 80% identity to SEQ ID NO: 3.

11. The method of claim 1, wherein the mSCF polypeptide comprises a sequence with at least 80% identity to 20 contiguous amino acids of SEQ ID NO: 1.

12. The method of claim 1, wherein the mSCF polypeptide comprises (i) a sequence with between 80% to 99% identity to 20 contiguous amino acids of SEQ ID NO: 1, and (ii) one or more amino acid substitutions as compared to said 20 contiguous amino acids of SEQ ID NO: 1.

13. (canceled)

14. The method of claim 1, wherein the mSCF polypeptide comprises a sequence with at least 80% identity to 20 contiguous amino acids of SEQ ID NO: 4.

15. The method of claim 1, wherein the mSCF polypeptide comprises a sequence with at least 80% identity to SEQ ID NO: 4.

16. The method of claim 1, wherein the mSCF polypeptide comprises the amino acid sequence of SEQ ID NO: 5.

17. The method of claim 1, wherein the treating comprises: (i) inhibiting hair loss, or (ii) promoting increased hair growth; as compared to the absence of said treating.

18. The method of claim 1, wherein the mSCF polypeptide is at least 20 amino acids in length; optionally wherein the mSCF polypeptide is at least 30 amino acids in length.

19. The method of claim 18, wherein the mSCF polypeptide is 20 amino acids to 75 amino acids in length; optionally wherein the mSCF polypeptide is about 30 amino acids in length.

20. The method of claim 1, wherein the mSCF polypeptide comprises at least 4 contiguous amino acids of SEQ ID NO: 29; optionally wherein the mSCF polypeptide comprises 5 to 13 contiguous amino acids of SEQ ID NO: 29.

21. The method of claim 20, wherein: (a) the mSCF polypeptide is selected from any one of SEQ ID NOs: 51-57; or (b) the mSCF polypeptide consists of the amino acid sequence according to SEQ ID NO: 54, 55, or 57.

22. (canceled)

23. The method of claim 1, wherein the mSCF polypeptide comprises one or more of a biotin group, a palmitoyl group, a norleucine, or an aspartic acid methyl ester; optionally wherein the biotin group is joined to the mSCF polypeptide by a C11 linker.

24. The method of claim 1, wherein:
(a) the mSCF polypeptide is joined to a cell-penetrating peptide; optionally wherein the cell-penetrating peptide comprises the sequence of SEQ ID NO: 6 or 7; or
(b) the mSCF polypeptide is joined to a cell-penetrating peptide by a linker sequence; optionally wherein the linker sequence comprises the sequence of SEQ ID NO: 8.

25. (canceled)

26. The method of claim 1, wherein the composition further comprises an MSH polypeptide; optionally wherein the MSH polypeptide is a β-MSH polypeptide or a γ-MSH polypeptide.

27. The method of claim 1, wherein the composition further comprises an Endothelin polypeptide; optionally wherein the Endothelin polypeptide is an Endothelin-1 polypeptide or an Endothelin-3 polypeptide.

28. The method of claim 1, wherein the composition further comprises a delivery vehicle, wherein the delivery vehicle is effective to increase delivery of the mSCF polypeptide across the epidermis of the human subject.

29. (canceled)

30. (canceled)

31. A composition comprising an mSCF polypeptide, wherein (i) the mSCF polypeptide is joined to a cell-penetrating peptide, or (ii) the mSCF polypeptide comprises an N-terminal truncation of at least 10 amino acids as compared to SEQ ID NO: 1, such that the mSCF polypeptide does not comprise 50 contiguous amino acids with more than 80% identity to SEQ ID NO: 2.

32.-57. (canceled)

58. A polynucleotide encoding the mSCF polypeptide according to claim 31.

59. A cell comprising the polynucleotide according to claim 58.
